SEQUENCE LISTING (23 sequences)

SEQ ID NO: 1 - protein, 351 aa, Artificial Sequence
  Amino acid sequence RTP13341, TALE995
MAYPYDVPDYAYPYDVPDYAYPYDVPDYAMDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQALRLFSGCCRFSAKHTV

SEQ ID NO: 2 - protein, 589 aa, Artificial Sequence
  Amino acid sequence RTP13336
MAYPYDVPDYAYPYDVPDYAYPYDVPDYAMDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNKGGKQALETVQRLLPVLCQAHGLTPEQVVAIASNSGGKQALETVQRLLPVLCQAHGLTPEQVVAIASNIGGKQAWRRYSACFPSFAKLTG

SEQ ID NO: 3 - protein, 487 aa, Artificial Sequence
  Amino acid sequence RTP13346
MAYPYDVPDYAYPYDVPDYAYPYDVPDYAMDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALKRSSVCSLSSVRHTV

SEQ ID NO: 4 - DNA, 2978 bp, Artificial Sequence
  CDS RTP13341
atggcttacccgtacgacgtgcctgactacgcctacccttacgacgtacccgactacgcctatccttacgacgttcccgactacgccatggacccgatccgatcccgtaccccgtccccggcccgggagttgctgccgggtccgcaacctgatagggtacaacccaccgccgataggggtggcgcaccacctgccggaggcccacttgacgggttgcccgcacgaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggacaatgtcaaggacccggctgccttcgccaccggctccaagcccagccttcagtgccggatccttctcggacttgctccgtcagttcgacccttccctcctggacacctccctactggactcgatgcctgccgtgggaacccctcacacagccgcagctcccgcagagtgggacgaagtgcaatccggcctgagagccgctgacgaccccccgcctaccgtgagggtagccgtaaccgccgcaagacctccgagggcaaagcctgcaccaaggcgaagggctgcccaaccttctgacgcctcaccggctgcccaagtggacctgcgaacactgggctactcccaacagcagcaagagaagatcaagccgaaggtccgaagcaccgtggcccaacaccacgaggccctagtgggtcacggctttacccacgcccatatcgtggccctctcccaacacccggccgcgctaggcacagttgccgtgaagtaccagcatattatcaccgctctgcctgaggccacccacgaggatatcgttggcgtcgggaagcagtggagcggagcccgagcactcgaagctctactgaccaaggctggcgagctgaggggtcctccgctacagctagacaccggccagcttctgaagatcgcaaagcgtggcggagtgaccgccgtcgaagcggttcacgcgtggcgcaacgccttgactggtgcaccgctgaacctgaccccggagcaagtcgtagcgatcgcttccaactcgggagggaaacaggccctgagactgttcagcggttgctgccggttctctgccaagcacacggtttgactcccgagcaagtggttgctatcgcgtccaacggaggagggaagcaagccctggaaacggttcaaaggctgcttcccgtcctctgtcaagctcacggacttacgccggagcaagtggttgcaattgctagcaacaaaggcggcaagcaagccctagaaaccgtccaaaggctccttcccgttctctgccaagctcacggtttgacaccggagcaagtggtggctatagcgtccaatattggcgggaagcaagccctcgagactgttcagcggttgctgccggttctctgccaagcacacggtttgactcccgagcaagtggttgctatcgcgtccaacggaggagggaagcaagccctcgagactgttcagcggttgctgccggttctctgccaagcacacggtttgactcccgagcaagtggttgctatcgcgtccaacggaggagggaagcaagccctagaaaccgtccaaaggctccttcccgttctctgccaagctcacggtttgacaccggagcaagtggtggctatagcgtccaatattggcgggaagcaagcccttgagacagttcagcgcttgcttcccgttctgtgccaagcacacggtcttacccctgagcaagtggtcgcgatagcttcgcacgacggtgggaagcaagccctggaaacggttcaaaggctgcttcccgtcctctgtcaagctcacggacttacgccggagcaagtggttgcaattgctagcaacaaaggcggcaagcaagccctggagacggtacagcgcttgcttcccgtcctttgccaagctcacgggctgacacccgagcaagtcgtggctatcgcgtctaactccggcgggaaacaggcgctggagtcgatcgtcgctcaactgagccgtccggaccctgcactggctgccttgaccaacgaccacctggtagccctcgcctgcctgggcggacgaccggcgctggacgcggtgaagaaggggctcccacacgcaccggagctgatccgccggattaatcgccggataccggagaggacctctcaccgagtgccggacctggctcacgtagtccgagtgctgggcttcttccaaagccactcccaccctgcacaagccttcgacgacgcgatgacccagttcgagatgtcacgacacggcctggtgcaactgttccgacgggtaggcgtgacggaattcgaagcgcgctacggcacactgccaccggcctcccagaggtgggataggatactgcaagcctccggtatgaagcgagccaagccgtcaccgacttccgctcagacgccggatcaggcctccctccacgcctttgccgactccctggagagggacctggacgccccgagccctatgcacgaaggagaccagacccgtgcctcctcgaggaagaggtccaggtctgatagggctgtaaccggcccgagcacccagcagagcttcgaagtgagggtgccggaacagcaagacgccctgcacctgcctctgtcctggcgcgtgaagaggccaaggacccgtataggcggcggactgccagatcctggaaccccgatcgcagccgatctagctgcgagctcaaccgtgatgtgggagcaagacgctgcaccgtttgctggcgctgccgacgatttccccgccttcaacgaggaagagttggcctggctgatggagctgcttccgcaatccggctccgtaggtggaacaatttga

SEQ ID NO: 5 - DNA, 2876 bp, Artificial Sequence
  CDS RTP13336
atggcttacccgtacgacgtgcctgactacgcctacccttacgacgtacccgactacgcctatccttacgacgttcccgactacgccatggacccgatccgatcccgtaccccgtccccggcccgggagttgctgccgggtccgcaacctgatagggtacaacccaccgccgataggggtggcgcaccacctgccggaggcccacttgacgggttgcccgcacgaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggacaatgtcaaggacccggctgccttcgccaccggctccaagcccagccttcagtgccggatccttctcggacttgctccgtcagttcgacccttccctcctggacacctccctactggactcgatgcctgccgtgggaacccctcacacagccgcagctcccgcagagtgggacgaagtgcaatccggcctgagagccgctgacgaccccccgcctaccgtgagggtagccgtaaccgccgcaagacctccgagggcaaagcctgcaccaaggcgaagggctgcccaaccttctgacgcctcaccggctgcccaagtggacctgcgaacactgggctactcccaacagcagcaagagaagatcaagccgaaggtccgaagcaccgtggcccaacaccacgaggccctagtgggtcacggctttacccacgcccatatcgtggccctctcccaacacccggccgcgctaggcacagttgccgtgaagtaccagcatattatcaccgctctgcctgaggccacccacgaggatatcgttggcgtcgggaagcagtggagcggagcccgagcactcgaagctctactgaccaaggctggcgagctgaggggtcctccgctacagctagacaccggccagcttctgaagatcgcaaagcgtggcggagtgaccgccgtcgaagcggttcacgcgtggcgcaacgccttgactggtgcaccgctgaacctgaccccggagcaagtcgtagcgatcgcttccaactcgggagggaaacaggccctagaaaccgtccaaaggctccttcccgttctctgccaagctcacggtttgacaccggagcaagtggtggctatagcgtccaatattggcgggaagcaagccctcgagactgttcagcggttgctgccggttctctgccaagcacacggtttgactcccgagcaagtggttgctatcgcgtccaacggaggagggaagcaagcccttgagacagttcagcgcttgcttcccgttctgtgccaagcacacggtcttacccctgagcaagtggtcgcgatagcttcgcacgacggtgggaagcaagccctcgagactgttcagcggttgctgccggttctctgccaagcacacggtttgactcccgagcaagtggttgctatcgcgtccaacggaggagggaagcaagccctggaaacggttcaaaggctgcttcccgtcctctgtcaagctcacggacttacgccggagcaagtggttgcaattgctagcaacaaaggcggcaagcaagcccttgaaacggtccagcgtctgctccctgtcctctgtcaggcacacggtctgacccctgagcaagtggtggctatcgcgagcaattcgggaggcaaacaagccctagaaaccgtccaaaggctccttcccgttctctgccaagctcacggtttgacaccggagcaagtggtggctatagcgtccaatattggcgggaagcaagcctggagacggtacagcgcttgcttcccgtcctttgccaagctcacgggctgacacccgagcaagtcgtggctatcgcgtctaactccggcgggaaacaggcgctggagtcgatcgtcgctcaactgagccgtccggaccctgcactggctgccttgaccaacgaccacctggtagccctcgcctgcctgggcggacgaccggcgctggacgcggtgaagaaggggctcccacacgcaccggagctgatccgccggattaatcgccggataccggagaggacctctcaccgagtgccggacctggctcacgtagtccgagtgctgggcttcttccaaagccactcccaccctgcacaagccttcgacgacgcgatgacccagttcgagatgtcacgacacggcctggtgcaactgttccgacgggtaggcgtgacggaattcgaagcgcgctacggcacactgccaccggcctcccagaggtgggataggatactgcaagcctccggtatgaagcgagccaagccgtcaccgacttccgctcagacgccggatcaggcctccctccacgcctttgccgactccctggagagggacctggacgccccgagccctatgcacgaaggagaccagacccgtgcctcctcgaggaagaggtccaggtctgatagggctgtaaccggcccgagcacccagcagagcttcgaagtgagggtgccggaacagcaagacgccctgcacctgcctctgtcctggcgcgtgaagaggccaaggacccgtataggcggcggactgccagatcctggaaccccgatcgcagccgatctagctgcgagctcaaccgtgatgtgggagcaagacgctgcaccgtttgctggcgctgccgacgatttccccgccttcaacgaggaagagttggcctggctgatggagctgcttccgcaatccggctccgtaggtggaacaatttga

SEQ ID NO: 6 - DNA, 3080 bp, Artificial Sequence
  CDS RTP13346
atggcttacccgtacgacgtgcctgactacgcctacccttacgacgtacccgactacgcctatccttacgacgttcccgactacgccatggacccgatccgatcccgtaccccgtccccggcccgggagttgctgccgggtccgcaacctgatagggtacaacccaccgccgataggggtggcgcaccacctgccggaggcccacttgacgggttgcccgcacgaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggacaatgtcaaggacccggctgccttcgccaccggctccaagcccagccttcagtgccggatccttctcggacttgctccgtcagttcgacccttccctcctggacacctccctactggactcgatgcctgccgtgggaacccctcacacagccgcagctcccgcagagtgggacgaagtgcaatccggcctgagagccgctgacgaccccccgcctaccgtgagggtagccgtaaccgccgcaagacctccgagggcaaagcctgcaccaaggcgaagggctgcccaaccttctgacgcctcaccggctgcccaagtggacctgcgaacactgggctactcccaacagcagcaagagaagatcaagccgaaggtccgaagcaccgtggcccaacaccacgaggccctagtgggtcacggctttacccacgcccatatcgtggccctctcccaacacccggccgcgctaggcacagttgccgtgaagtaccagcatattatcaccgctctgcctgaggccacccacgaggatatcgttggcgtcgggaagcagtggagcggagcccgagcactcgaagctctactgaccaaggctggcgagctgaggggtcctccgctacagctagacaccggccagcttctgaagatcgcaaagcgtggcggagtgaccgccgtcgaagcggttcacgcgtggcgcaacgccttgactggtgcaccgctgaacctgaccccggagcaagtcgtagcgatcgcttccaactcgggagggaaacaggccctagaaaccgtccaaaggctccttcccgttctctgccaagctcacggtttgacaccggagcaagtggtggctatagcgtccaatattggcgggaagcaagccctcgagactgttcagcggttgctgccggttctctgccaagcacacggtttgactcccgagcaagtggttgctatcgcgtccaacggaggagggaagcaagccctcgagactgttcagcggttgctgccggttctctgccaagcacacggtttgactcccgagcaagtggttgctatcgcgtccaacggaggagggaagcaagccctcgagactgttcagcggttgctgccggttctctgccaagcacacggtttgactcccgagcaagtggttgctatcgcgtccaacggaggagggaagcaagccctgaaacggtccagcgtctgctccctgtcctctgtcaggcacacggtctgacccctgagcaagtggtggctatcgcgagcaattcgggaggcaaacaagccctcgagactgttcagcggttgctgccggttctctgccaagcacacggtttgactcccgagcaagtggttgctatcgcgtccaacggaggagggaagcaagccctggaaacggttcaaaggctgcttcccgtcctctgtcaagctcacggacttacgccggagcaagtggttgcaattgctagcaacaaaggcggcaagcaagccctggaaacggttcaaaggctgcttcccgtcctctgtcaagctcacggacttacgccggagcaagtggttgcaattgctagcaacaaaggcggcaagcaagccctggaaacggttcaaaggctgcttcccgtcctctgtcaagctcacggacttacgccggagcaagtggttgcaattgctagcaacaaaggcggcaagcaagccctggagacggtacagcgcttgcttcccgtcctttgccaagctcacgggctgacacccgagcaagtcgtggctatcgcgtctaactccggcgggaaacaggcgctggagtcgatcgtcgctcaactgagccgtccggaccctgcactggctgccttgaccaacgaccacctggtagccctcgcctgcctgggcggacgaccggcgctggacgcggtgaagaaggggctcccacacgcaccggagctgatccgccggattaatcgccggataccggagaggacctctcaccgagtgccggacctggctcacgtagtccgagtgctgggcttcttccaaagccactcccaccctgcacaagccttcgacgacgcgatgacccagttcgagatgtcacgacacggcctggtgcaactgttccgacgggtaggcgtgacggaattcgaagcgcgctacggcacactgccaccggcctcccagaggtgggataggatactgcaagcctccggtatgaagcgagccaagccgtcaccgacttccgctcagacgccggatcaggcctccctccacgcctttgccgactccctggagagggacctggacgccccgagccctatgcacgaaggagaccagacccgtgcctcctcgaggaagaggtccaggtctgatagggctgtaaccggcccgagcacccagcagagcttcgaagtgagggtgccggaacagcaagacgccctgcacctgcctctgtcctggcgcgtgaagaggccaaggacccgtataggcggcggactgccagatcctggaaccccgatcgcagccgatctagctgcgagctcaaccgtgatgtgggagcaagacgctgcaccgtttgctggcgctgccgacgatttccccgccttcaacgaggaagagttggcctggctgatggagctgcttccgcaatccggctccgtaggtggaacaatttga

SEQ ID NO: 7 - protein, 307 aa, Artificial Sequence
  RTP13341 without HA TAG and unnatural amino acids
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQAL

SEQ ID NO: 8 - protein, 544 aa, Artificial Sequence
  RTP13336 without HA TAG and unnatural amino acids
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNKGGKQALETVQRLLPVLCQAHGLTPEQVVAIASNSGGKQALETVQRLLPVLCQAHGLTPEQVVAIASNIGGKQA

SEQ ID NO: 9 - protein, 443 aa, Artificial Sequence
  RTP13346 without HA TAG and unnatural amino acids
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPEQVVAIASNSGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQAL

SEQ ID NO: 10 - protein, 1446 aa, Artificial Sequence
  Amino acid sequence of AvrXa7
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTEARELRGPPLQLDTGQLLKIAKRGGVTAVEAVHAWRNALTGAPLNLTPDQVVAIASNIGGKQALETVQRLLPVLCQDHGLTPDQVVAIASHGGGKQALETVQRLLPVLCQDHGLTPDQVVAIASNIGGKQALETVQRLLPVLCQAHGLTPDQVVAIASNIGGKQALETVQRLLPVLCQDHGLTPAQVVAIASNSGGKQALETVQRLLPVLCQDHGLTPDQVVAIASHDGGKQALETVQRLLPVLCQDHGLTPDQVVAIANNNGGKQALETLQRLLPVLCQDHGLTPDQVVAIASHDGGKQALETVQRLLPVLCQDHGLTPDQVVAIASHDGGKQALETVQRLLPVLCQDHGLTPAQVVAIASHDGGKQALETVQRLLPVLCQDHGLTPDQVVAIASNSGGKQALETVQRLLPVLCQDHGLTPDQVVAIASNGGKQALETVQRLLPVLCQDHGLTPDQVVAIASNGGKQALETVQRLLPVQRLLPVLCQDHGLTQDQVVAIASHDGGKQALETVQRLLPVLCQDHGLTPDQVVAIASHDGGKQALETVQRLLPVLCQDHGLTPDQVVAIASNSGGKQALETVQRLLPVLCQDHGLTPDQVVAIASNSGGKQALETVQRLLPVLCQDHGLTPDQVVAIASNNGGKQALETVQRLLPVLCQDHGLTPDQVVAIANNNGGKQALETVQRLLPVLCQDHGLTPAQVVAIASNIGGKQALETVQRLLPVLCQDHGLTLDQVVAIASNGGSKQALETVQRLLPVLCQDHGLTPDQVVAIANNNGGKQALETVQRLLPVLCQDHGLTPDQVVAIASNIGGKQALETVQRLLPVLCQDHGLTLDQVVAIASNGGKQALETVQRLLPVLCQDHGLTPNQVVAIASNSGGKQALETVQRLLPVLCQDHGLTPNQVVAIASNGGKQALESIVAQLSRPDPALAALTNDHLVALACLGGRPALDAVKKGLPHAPELIRRINRRIPERTSHRVPDLAHVVRVLGFFQSHSHPAQAFDDAMTQFEMSRHGLVQLFRRVGVTELEARGGTLPPASQRWDRILQASGMKRAKPSPTSAQTPDQASLHAFADSLERDLDAPSPMHEGDQTGASSRKRSRSDRAVTGPSAQQSFEVRVPEQRDALHLPLSWRVKRPRTRIGGGLPDPGTPIAADLAASSTVMWEQDAAPFAGAADDFPAFNEEELAWLMELLPQSGSVGGTI

SEQ ID NO: 11 - protein, 9 aa, Artificial Sequence
  Amino acid sequence, 1xHA tag
YPYDVPDYA

SEQ ID NO: 12 - protein, 27 aa, Artificial Sequence
  Amino acid sequence, 3xHA tag
YPYDVPDYAYPYDVPDYAYPYDVPDYA

SEQ ID NO: 13 - DNA, 81 bp, Artificial Sequence
  nucleic acid sequence, 3xHA tag
tacccgtacgacgtgcctgactacgcctacccttacgacgtacccgactacgcctatccttacgacgttcccgactacgcc

SEQ ID NO: 14 - protein, 15 aa, Artificial Sequence
  sequence added by frameshift, mentioned in Example 1
RLFSGCCRFSAKHTV

SEQ ID NO: 15 - protein, 11 aa, Artificial Sequence
  Sequence in Figure 1
TNACGCGGGAN

SEQ ID NO: 16 - protein, 9 aa, Artificial Sequence
  Sequence in Figure 1
TNATCTGNA

SEQ ID NO: 17 - protein, 152 aa, Artificial Sequence
  T3S and translocation signal
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQ

SEQ ID NO: 18 - protein, 287 aa, Artificial Sequence
  AvrXa10 partial sequence from Figure 8
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAECDEVQSGLRAADDPPPTVRVAVTARPPRAKPAPRRRAAQPSDASPAAQVDLRTLGYSQQQQEKIKPKVRSTVAQHHEALVGHGFTHAHIVALSQHPAALGTVAVTYQDIIRALPEATHEDIVGVGKQWSGARALEALLTEAGELRGPPLQLDTGQLLKIAKRGGVTAVEAVHAWRNALTGAPLN

SEQ ID NO: 19 - protein, 288 aa, Artificial Sequence
  AvrXa7, partial sequence from Figure 8
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTEARELRGPPLQLDTGQLLKIAKRGGVTAVEAVHAWRNALTGAPLN

SEQ ID NO: 20 - protein, 288 aa, Artificial Sequence
  AvrBs3, partial sequence from Figure 8
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 288 aa, Artificial Sequence
  Hax3, partial sequence from Figure 8
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 22 - protein, 34 aa, Artificial Sequence
  Tandem repeat sequence
  VARIANT (12)..(13): Xaa is any amino acid
LTPDQVVAIASXXGGKQALETVQRLLPVLCQDHG

SEQ ID NO: 23 - protein, 18 aa, Artificial Sequence
  partial Tandem repeat sequence
TPEQVVAIASNSGGKQAL

(19) United States
(12) Patent Application Publication
(10) Pub. No.: US 2024/0110193 A1
(43) Pub. Date: Apr. 4, 2024

(54) INCREASE IN CORN TRANSFORMATION EFFICACY BY THE N-TERMINUS OF A TALE

(71) Applicant: BASF PLANT SCIENCE COMPANY GMBH, Ludwigshafen (DE)

(72) Inventors: Thaquoris Pitt, Research Triangle Park, NC (US); Sean McAdams, Research Triangle Park, NC (US); Minesh Patel, Research Triangle Park, NC (US); Mary F. Dubois, Snow Camp, NC (US); Leendert W. Neuteboom, Youngsville, NC (US)

(21) Appl. No.: 17/767,580

(22) PCT Filed: Oct. 9, 2020
(86) PCT No.: PCT/EP2020/078451

§ 371 Date: Apr. 8, 2022

(30) Foreign Application Priority Data
Oct. 11, 2019 (EP) 19202802.5

(51) Int. Cl. C12N 15/82; C07K 14/195

(52) U.S. Cl. CPC C12N 15/8205; C07K 14/195

(57) ABSTRACT

The present invention is in the field of molecular biology and relates to improved methods for plant transformation and to polynucleotides and polypeptides for achieving the same.

### FIELD OF THE INVENTION

The present invention is in the field of molecular biology and relates to improved methods for plant transformation and to polynucleotides and polypeptides for achieving the same.

### BACKGROUND SECTION

Plant transformation is now widely used for basic research as well as for generation of commercially used transgenic crops. Transgenic plants are typically produced by complex methods, for example by Agrobacterium-mediated transformation. Agrobacterium is a naturally occurring pathogenic soil bacterium which is capable of transferring DNA into the genome of plant cells. For Agrobacterium-mediated plant transformation, the gene of interest is placed between the left and right border repeats of Agrobacterium T-DNA (transfer DNA). Afterwards, the T-DNA region containing the gene of interest is stably integrated into the plant genome by using an appropriate plant transformation protocol (for a review see Gelvin, 2003 Microbiol Mol Biol Rev. 67(1): 16-37). Aside from Agrobacterium-mediated plant transformation, other plant transformation methods exist such as viral transformation, electroporation of plant protoplasts, and particle bombardment.

Generally, plant transformation techniques are based on the same principles. In a first step, the gene of interest is introduced in a suitable transformation vector. The transformation vector harboring the gene of interest is then introduced into regenerable cells of a target plant. Since only a minor proportion of target cells receive the gene of interest, selection for transformed plant cells among a large excess of untransformed cells is carried out. Moreover, once the gene of interest has been stably introduced into the genome of a host cell, it is essential to establish regeneration conditions to regenerate whole plants from a single transformed plant cell (see, e.g., Birch, 1997, Annu. Rev. Plant Physiol. Plant Mol. Biol. 48: 297-326).

Regeneration of whole transformed plants is considered as one bottleneck in plant transformation since regeneration is very time-consuming. Transformation procedures that would allow for a faster generation of transgenic plants are, therefore, highly desired. Further, transformation procedures which allow for a higher transformation efficacy are highly desired.

TAL effectors (TALEs) of plant pathogens belonging to the genus of Xanthomonas are important virulence factors that bind specific sequences in the plant genome resulting in altered gene expression in the host cell to support bacterial proliferation. DNA-binding is made possible through the central repeat region (abbreviated “CRR”), a central region of polymorphic repeats. This central repeat region consists of multiple, tandemly arranged, ˜34 amino acid repeat motifs, which are hypervariable in the amino acids at position 12 and 13 (the Repeat Variable Diresidue or RVD). Each repeat was discovered to bind one bp in a contiguous target sequence in a neighbor-independent manner, with the RVD sequence determining the specificity of binding. The cipher governing the specificity was found to be surprisingly simple: RVD sequence NI-binds A, HD-binds C, NG-binds T, NK-binds G and NS-binds all 4 nucleotides (Scholze and Boch, 2011). The last repeat is only a half repeat, but contains the two hyper-variable residues and can be designed to bind any nucleotide. The C-terminus of TALE contains nuclear localization signals (NLS) and the transcriptional activation domain (AD). A TALE requires the presence of at least 8 units in the DNA binding domain to meet the minimal affinity for transcriptional activation (Boch et al., 2009) and amino acids after the half repeat are required for proper folding of the DNA binding domain (Zhang et al., 2011, Christian et al., 2010, Miller et al., 2011, Mussolino et al., 2011, Sun et al., 2012).

The N-terminal domain of TAL effectors comprises a type III secretion signal and four “cryptic” repeats (repeat −3, repeat −2, repeat −1 and repeat 0) that structurally resemble the central repeats. These cryptic repeats are posited to nucleate binding to DNA. The four cryptic repeats play a less specific but crucial role in the DNA interaction. In general, TALEs demonstrate preference for a thymine (T) nucleotide at the 5′ position immediately preceding the target site. Therefore, repeat 0, i.e. the cryptic repeat immediately before the first repeat of the CRR is frequently also referred to as TO repeat (Read et al., 2016, Triplett et al., 2016).

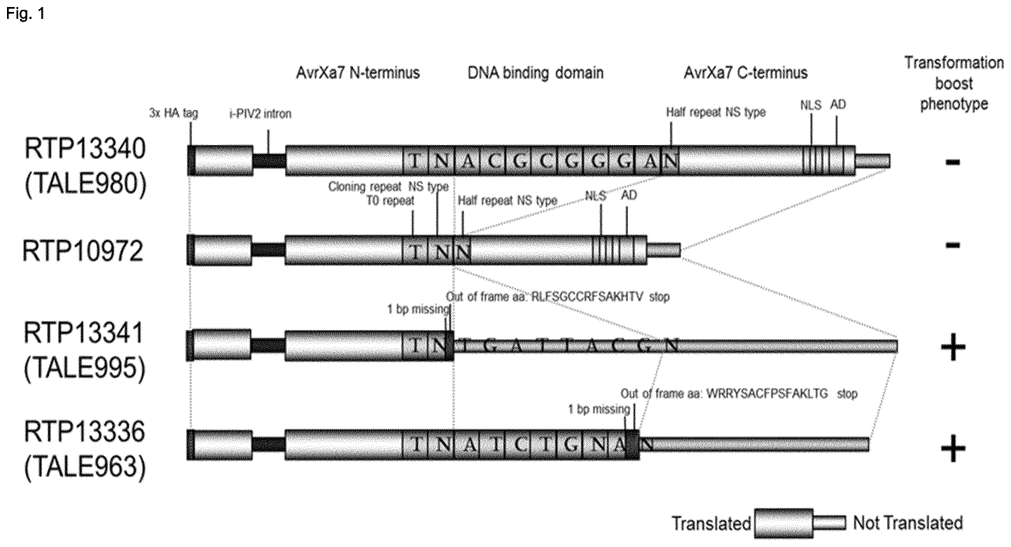

Schornack et al. showed that truncated TALEs (AvrBs4 type) triggers HR (hypersensitive response) mediated by Bs4 in tomato. When delivered through Agrobacterium the truncated some, but not all truncated TALEs triggered Bs4 dependent HR as did the full length AvrBs4 (see FIG. 1 of Schornack). The tested truncated TALEs lacked a complete CDR domain and the entire CTR which includes the NLSs and AD. In another paper, Schornack et al. (2005) showed that Bs4 expression is constitutive regardless of the presence of Xanthomonas.

Triplett et al. (2016) showed that truncated TALEs also induce HR in rice. However, truncated TALEs with a CRR having 0.5, 1.5 and 2.5 repeats did induce HR.

Read et al. (2016) showed that truncated TALEs suppress resistance mediated by the Xo1 locus.

Ji et al. (2016) show that two truncated TALE effectors, Tal3a and Tal3b interfere with Xa1-mediated resistance in rice.

Although significant advances have been made in the field of transformation methods, a need continues to exist for improved methods to facilitate the ease, speed and efficiency of such methods for transformation of plants. Therefore, it was the objective of the present invention to provide an improved method having higher overall efficiency in the process of generation of transgenic plants. This objective is solved by the present invention.

Surprisingly, it was shown in the studies underlying the present invention that introduction of a truncated transcription activator-like effector (TALE) polypeptide allows for a general improvement of transformation. The truncated TALE polypeptide comprises the N-terminus of a TALE polypeptide. In particular, the introduction allowed for an enhanced transformation efficacy, a faster growth of transformed calli, a faster generation of TO plants. Further, an increased biomass of generated TO plants was observed. Advantageously, in the T1 generation plants appeared to develop normally. The transformation efficacy was increased more than 2 fold over controls and plants were moved to the greenhouse 3 weeks earlier. The observed effect was independent of nuclear localization and transcriptional activation as both corresponding domains are not functionally present in the truncated protein.

### BRIEF SUMMARY OF THE PRESENT INVENTION

The present invention relates to method for generating a transgenic plant comprising at least one polynucleotide of interest, the method comprising
(a) providing
   (i) a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
   and/or
   (ii) a polynucleotide encoding the truncated transcription activator-like effector (TALE) polypeptide of (i), or the functional variant thereof,
(b) providing at least one polynucleotide of interest,
(c) introducing the polypeptide or polynucleotide provided in step (a), and the at least one polynucleotide of interest provided in step (b) into a plant cell, and
(d) regenerating a transgenic plant comprising the at least one polynucleotide of interest from said plant cell.

In an embodiment of the aforementioned method, the method comprises the steps of
(a) providing a polynucleotide encoding a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
(b) providing at least one polynucleotide of interest,
(c) introducing polynucleotide provided in step (a), and the at least one polynucleotide of interest provided in step (b) into a plant cell, and
(d) regenerating a transgenic plant comprising the at least one polynucleotide of interest from said plant cell.

In an embodiment of the aforementioned methods, the polynucleotide provided in step (a), and the at least one polynucleotide of interest provided in step (b) are introduced in step c) into the plant cell by Agrobacterium mediated transformation. E.g., the polynucleotides are introduced simultaneously.

In an embodiment of the aforementioned methods, the polynucleotides provided in step (a), and the at least one polynucleotide of interest provided in step (b) are stably introduced in step c) into the plant cell by Agrobacterium mediated transformation.

In an embodiment of the aforementioned methods, the transgenic plant regenerated in step (d) further comprises the polynucleotide encoding the truncated transcription activator-like effector (TALE) polypeptide, or the functional variant thereof.

In an embodiment of the aforementioned methods, seeds are collected from the regenerated plants.

The present invention also contemplates a method for improving plant transformation comprising
(a) providing
   (i) a truncated transcription activator-like effector (TALE) polypeptide, or a functional variant of the truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
   and/or
   (ii) a polynucleotide encoding the truncated transcription activator-like effector (TALE) polypeptide of (i), or the functional variant thereof,
(b) providing at least one polynucleotide of interest, and
(c) introducing the polypeptide or polynucleotide provided in step (a) and the at least one polynucleotide of interest provided in step (b) into a plant, thereby improving plant transformation.

In an embodiment of the aforementioned method, the method for improving plant transformation comprises the steps of
(a) providing a polynucleotide encoding a truncated transcription activator-like effector (TALE) polypeptide, or a functional variant of the truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
(b) providing at least one polynucleotide of interest, and
(c) introducing polynucleotide provided in step (a) and the at least one polynucleotide of interest provided in step (b) into a plant, thereby improving plant transformation.

In an embodiment of the aforementioned methods for improving plant transformation, the transformation to be improved is Agrobacterium-mediated transformation.

In an embodiment of the aforementioned methods for improving plant transformation, the polynucleotide provided in step (a), and the at least one polynucleotide of interest provided in step (b) are introduced in step c) into the plant cell by Agrobacterium mediated transformation. E.g., the polynucleotides are introduced simultaneously.

In an embodiment of the aforementioned methods for improving plant transformation, the at least one polynucleotide of interest as provided in step b) is stably introduced into the plant by Agrobacterium-mediated transformation.

In an embodiment of the aforementioned methods for improving plant transformation, the at least one polynucleotide of interest as provided in step b) and the polynucleotide provided in step (a) (ii) are stably introduced into the plant by Agrobacterium-mediated transformation.

In an embodiment of the aforementioned methods for improving plant transformation, the truncated TALE polypeptide, or functional variant thereof, or the polynucleotide encoding said truncated polypeptide, or functional variant thereof, is transiently introduced into the plant.

In an embodiment of the aforementioned methods for improving plant transformation or for generating a plant, the polynucleotide encoding said truncated TALE polypeptide, or functional variant thereof, as provided in step (a)(ii), and the at least one polynucleotide of interest as provided in step b) are present in the same T-DNA or in different T-DNAs.

In an embodiment of the aforementioned methods for improving plant transformation, the improvement of plant transformation
(i) enhanced transformation efficacy,
(ii) a faster growth of transformed calli,
(iii) a faster generation of TO plants, and/or
(iv) an increased biomass of generated TO plants.

The present invention also relates to a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete TALE Central Repeat Region (CRR), and wherein said truncated TALE polypeptide does not comprise a transcriptional activation domain.

The present invention also relates to a polynucleotide encoding for the truncated TALE polypeptide of the present invention, or functional variant thereof.

The present invention also relates to an expression vector comprising the polynucleotide of the present invention, operably linked to a heterologous promoter.

The present invention also relates to a host cell comprising the truncated TALE polypeptide of the present invention, the polynucleotide of the present invention, or the expression vector of the present invention.

In an embodiment, the host cell is an Agrobacterium cell or a plant cell.

In an embodiment, the host cell further comprising at least one polynucleotide of interest.

The present invention also relates to a plant comprising the truncated TALE polypeptide of the present invention, the polynucleotide of the present invention, or the expression vector of the present invention.

The present invention also relates to the use of the truncated TALE polypeptide of the present invention, the polynucleotide of the present invention, or the expression vector of the present invention, for improving plant transformation.

In an embodiment of the methods, the truncated TALE polypeptide (or the functional variant thereof), the polynucleotide, the expression vector, the host cell, the plant, or the use of the present invention, the truncated TALE polypeptide does not comprise nuclear localization signals (NLSs)

In an embodiment of the methods, the truncated TALE polypeptide (or the functional variant thereof), the polynucleotide, the expression vector, the host cell, the plant, or the use of the present invention, the N-terminal region of the TALE polypeptide comprises the T3S and translocation signal and repeats −3, −2, −1 and 0 of a TALE polypeptide.

In an embodiment of the methods, the truncated TALE polypeptide (or the functional variant thereof), the polynucleotide, the expression vector, the host cell, the plant, or the use of the present invention, the N-terminal region of the truncated TALE polypeptide comprises amino acids 1 to 288 of SEQ ID NO: 10.

In an embodiment of the methods, the truncated TALE polypeptide (or the functional variant thereof), the polynucleotide, the expression vector, the host cell, the plant, or the use of the present invention, the truncated TALE polypeptide comprises an incomplete Central Repeat Region (CRR).

In an embodiment of the methods, the truncated TALE polypeptide (or the functional variant thereof), the polynucleotide, the expression vector, the host cell, the plant, or the use of the present invention, the incomplete Central Repeat Region (CRR) comprises between 0.5 and 20 repeats, such as between 0.5 and 10 repeats, between 0.5 repeats and 8 repeats, between 0.5 and 6 repeats, between 0.5 and 5 repeats, between 0.5 and 4 repeats, between 0.5 and 3 repeats, between 0.5 and 2 repeats, or between 0.5 and 1 repeats.

In an embodiment of the methods, the truncated TALE polypeptide (or the functional variant thereof), the polynucleotide, the expression vector, the host cell, the plant, or the use of the present invention, the incomplete Central Repeat Region comprises or consists of the amino acid sequence Itpeqvvaiasnsggkqal (SEQ ID NO: 23)

In an embodiment of the methods, the truncated TALE polypeptide (or the functional variant thereof), the polynucleotide, the expression vector, the host cell, the plant, or the use of the present invention, the truncated TALE polypeptide is truncated within repeats 1 to 20, repeats 1 to 15, repeats 1 to 10, repeats 1 to 8, repeats 1 to 6, repeats 1 to 5, repeats 1 to 4, repeats 1 to 3, repeats 1 to 2, or within repeat 1 of the CRR of the TALE polypeptide.

In an embodiment of the present invention (i.e. of the methods, the truncated TALE polypeptide (or the functional variant thereof), the polynucleotide, the expression vector, the host cell, the plant, or the use of the present invention), the truncated transcription activator-like effector (TALE) polypeptide is derived from a TALE polypeptide from the genus Xanthomonas, e.g. from Xanthomonas oryzae.

In an embodiment of the present invention, the truncated transcription activator-like effector (TALE) polypeptide is derived from a TALE polypeptide selected from the group consisting of AvrXa7, AvrBs3, Hax3, PthXo6, AvrXa27, AvrRxo1, PthXo1, Hax2, and Hax4.

In an embodiment of the present invention, the truncated TALE polypeptide, or functional variant thereof has an amino acid sequence comprising or consisting of
(i) a sequence as shown in any one of SEQ ID Nos: 1, 2, 3, 7, 8, and 9,
(ii) a sequence comprising amino acids 1 to 307 of SEQ ID NO: 10
(iii) a sequence comprising amino acids 1 to 443 of SEQ ID NO: 10
(iv) a sequence comprising amino acids 1 to 544 of SEQ ID NO: 10, or
(v) a sequence which is at least 70% identical to the sequence under (i), (ii), (iii) or (iv).

In an embodiment of the present invention, the polynucleotide encoding said truncated TALE polypeptide, or functional variant thereof, comprises or consists of
(i) a sequence as shown in any one of SEQ ID Nos: 4 to 6, or
(ii) a sequence which is at least 70% identical to the sequence under i).

In an embodiment of the present invention, the functional variant of the truncated TALE polypeptide is capable of accelerating meristematic plant growth, when introduced into a plant.

In an embodiment of the present invention, the polynucleotide encoding the truncated TALE polynucleotide, or variant thereof, and the at least one polynucleotide of interest provided in step (b) are operably linked to a promoter.

In an embodiment of the present invention, the plant is a monocotyledonous plant.

In an embodiment of the present invention, the plant is a dicotyledonous plant.

In an embodiment of the present invention, the plant is not a tobacco plant.

In an embodiment of the present invention, the at least one polynucleotide of interest encodes a gene-editing polypeptide.

In an embodiment of the present invention, at least two polynucleotides of interest are provided in step b) and introduced into the plant cell in step b) of the methods of the present invention.

### DETAILED DESCRIPTION OF THE PRESENT INVENTION—DEFINITIONS

The present invention relates to a method for generating a transgenic plant comprising at least one polynucleotide of interest, the method comprising
(a) providing
   (i) a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
      and/or
   (ii) a polynucleotide encoding the truncated transcription activator-like effector (TALE) polypeptide of (i), or the functional variant thereof,
(b) providing at least one polynucleotide of interest,
(c) introducing the polypeptide or polynucleotide provided in step (a), and the at least one polynucleotide of interest provided in step (b) into a plant cell, and
(d) regenerating a transgenic plant comprising the at least one polynucleotide of interest from said plant cell.

In an embodiment, steps (a) to (d) of the method for regenerating a transgenic plant are as follows:
(a) providing a polynucleotide encoding a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
(b) providing at least one polynucleotide of interest,
(c) introducing polynucleotide provided in step (a), and the at least one polynucleotide of interest provided in step (b) into a plant cell, and
(d) regenerating a transgenic plant comprising the at least one polynucleotide of interest from said plant cell.

As set forth above, the present invention further relates to a method for improving plant transformation, the method comprising steps a) and b) of the method for generating a transgenic plant and step c) introducing the polypeptide or polynucleotide provided in step (a), and the at least one polynucleotide of interest provided in step (b) into a plant cell, thereby improving plant transformation. The definitions made in connection with the method of generating a transgenic plant apply mutatis mutandis to the method for improving plant transformation.

In step (a) of the methods of the present invention, i) a truncated transcription activator-like effector (abbreviated “TALE”) polypeptide, or a functional variant of said truncated TALE polypeptide, and/or ii) a polynucleotide encoding said truncated polypeptide, or the functional variant thereof, shall be provided. Said truncated TALE polypeptide shall comprise the N-terminal region of a TALE polypeptide, and optionally a complete or incomplete Central Repeat Region (CRR). However, said truncated TALE polypeptide shall not comprise, i.e. lack, a transcriptional activation domain.

As described herein below in detail, the polypeptide provided in step (a) shall be a truncated form of a TALE polypeptide, i.e. a fragment of a full-length TALE polypeptide. Full length TALE polypeptides are well known in the art and belong to the transcription activator-like (TAL) family of polypeptides (see e.g. Moore et al., 2014, and Scott et al., 2014 both of which are herewith incorporated by reference with respect to their entire disclosure content). Full-length TALE polypeptides are characterized by three conserved regions (see Cuculis et al., 2015):
an N-terminal region (NTR) which comprises the type III translocation system required for secretion and four cryptic repeats (repeat −3, repeat −2, repeat −1 and repeat 0),
a central repeat region (CRR, frequently also referred to as central repeat domain (CRD)) that forms specific DNA contacts, and
a C-terminal region (CTR) comprising one or more nuclear localization signals and an acidic activation domain.

Accordingly, a full-length TALE polypeptide comprises (from N-terminus to C-terminus) a N-terminal region (NTR), a central repeat region (CRR), and a C-terminal region (CTR).

Full length TALE polypeptides, but also some truncated TALE polypeptides, are expressed by bacterial plant pathogens, in particular of the genus Xanthomonas. Full length TALE polypeptides of the large TAL effector family are key virulence factors of Xanthomonas and reprogram host cells by mimicking eukaryotic transcription factors. TALE polypeptides are secreted by Xanthomonas bacteria via their type III secretion system when they infect various plant species. A functional TALE polypeptide can bind promoter sequences in the host plant and activate the expression of plant genes that aid bacterial infection. It recognizes plant DNA sequences through a central repeat domain consisting of a variable number of typically 34 amino acid tandem repeats. The tandem repeats of the CRR are herein also referred to as “repeats”. The amino acid sequences of the repeats are conserved, except for two adjacent highly variable residues (at positions 12 and 13) that determine specificity towards the DNA base A, G, C or T. The AAs in positions 12 and 13 are called repeat variable di-residue (RVD). Several TALEs of different strains are identical on the level of RVD sequences and are, hence, assumed to target the same plant genes, whereas other TALEs show variation in individual RVDs or even in longer, contiguous stretches of their RVD sequence. For example, a tandem repeat as set forth herein may have the following sequence: ItpdqvvaiasxxggkqaletvqrlIpvIcqdhg (SEQ ID NO: 22), wherein x can be any amino acid.

In an embodiment, the truncated TALE polypeptide as referred to herein is derived from a TALE polypeptide from the genus Xanthomonas, i.e. is a TALE polypeptide from the genus Xanthomonas. More preferably, the truncated TALE polypeptide is derived from a TALE polypeptide from a Xanthomonas species selected from the group of Xanthomonas species consisting of X. albilineans, X. alfalfae, X. ampelina, X. arboricola, X. axonopodis, X. boreopolis, X. badrii, X. bromi, X. campestris. X. cassavae, X. citri, X. codiaei, X. cucurbitae, X. cyanopsidis, X. cynarae, X. euvesicatoria, X. frageriae, X. gardneri, X. holcicola, X. hortorum, X. hyacinthi, X. malvacerum, X. maltophila, X. manihotis, X. melonis, X. oryzae, X. papavericola, X. perforans, X. phaseoli, X. pisi, X. populi, X. sacchari, X. theicola. X. translucens, X. vasicola, and X. vesicatoria. More preferably, the truncated TALE polypeptide is derived from a TALE polypeptide from Xanthomonas oryzae, in particular Xanthomonas oryzae pv. oryzae. Xanthomonas oryzae causes a serious blight of rice, other grasses and sedges.

The TALE polypeptide to be truncated as referred to in accordance with the present invention can be any TALE polypeptide. In an embodiment, the TALE polypeptide is selected from the group consisting of AvrXa7, AvrBs3, Hax3, PthXo6, AvrXa27, AvrRxo1, PthXo1, Hax2, and Hax4. In a more preferred embodiment, the truncated TALE polypeptide is a truncated AvrXa7 polypeptide. AvrXa7 is a TALE polypeptide from Xanthomonas oryzae pv. oryzae. Preferably, said AvrXa7 polypeptide has sequence as shown in SEQ ID NO: 10. The N-terminal region (NTR), the central repeat region (CRR), and the C-terminal region (CTR) of this polypeptide are as follows:
NTR: amino acids 1 to 288 of SEQ ID NO: 10
CRR: amino acids 289 to 1155 of SEQ ID NO: 10
CTR: amino acids 1156 to 1446 of SEQ ID NO: 10

The TALE polypeptide to be truncated can be a naturally occurring TALE polypeptide, as well as a non-naturally occurring TALE polypeptide. In an embodiment, the term “non-naturally occurring TALE polypeptide” refers to a naturally occurring TALE polypeptide which has been mutated. For example, mutations could be made within the DNA-binding domain. For example, the amino acids at positions 12 and/or 13 of the tandem repeats could be mutated (as compared to the naturally occurring amino acids at these positions).

As set forth above, the polypeptide provided in step a) shall be a truncated TALE polypeptide. Accordingly, the truncated TALE polypeptide is not a full-length TALE polypeptide. It comprises parts of a full-length TALE polypeptide, but lacks other parts of said TALE polypeptide. In particular, the term “truncated” refers to a truncated TALE polypeptide that lacks amino acids of at least the C-terminal region of a full-length TALE polypeptide, and thus to a TALE polypeptide having C terminal amino acids removed.

How to obtain a truncated polypeptide is well known in the art. For example, the nucleic acid sequence which encodes for the truncated polypeptide could be amplified by PCR (and cloned into a suitable vector). Also, said sequence could be produced by gene synthesis. Also, the nucleic acid sequence which encodes for parts of the TALE polypeptide which should not be present in the truncated TALE polypeptide can be removed from the polynucleotide which encodes for the full-length polynucleotide. Alternatively, artificial stop codons could be introduced at the position at which the polypeptide should be truncated.

In an embodiment, a TALE polypeptide, i.e. a full-length, TALE polypeptide, can be truncated in any region which results in a truncated polypeptide which comprises the N-terminal region as defined herein, but is devoid of the nuclear nucleation signal(s) and/or the transcriptional activation domain, yet retaining its plant transformation improving properties. In another embodiment, a TALE polypeptide, i.e. a full-length, TALE polypeptide, can be truncated in any region which results in a truncated polypeptide which comprises the N-terminal region as defined herein, but is devoid of the entire C-terminal region of the TALE polypeptide. In another embodiment, a TALE polypeptide, i.e. a full-length, TALE polypeptide, can be truncated in any region which results in a truncated polypeptide which comprises the N-terminal region as defined herein, but is devoid at least a portion of the CRR and of the entire C-terminal region of the TALE polypeptide.

In an embodiment, the truncated transcription activator-like effector (TALE) polypeptide comprises the N-terminal region (NTR) of a TALE polypeptide. Preferably, the N-terminal region of a TALE polypeptide comprises the T3S (type III S) and translocation signal and repeats −3, −2, −1 and 0 of a TALE polypeptide. The T3S (type III S) and translocation signal is herein also referred to as T3S region (see Cuculis et al., 2015). The repeat 0 is also known as TO repeat. Repeats −3, −2, −1 and 0 are so called cryptic repeats. Thus, the N-terminal region of a TALE polypeptide comprises the T3S (type III S) and translocation signal and four cryptic repeats of a TALE polypeptide

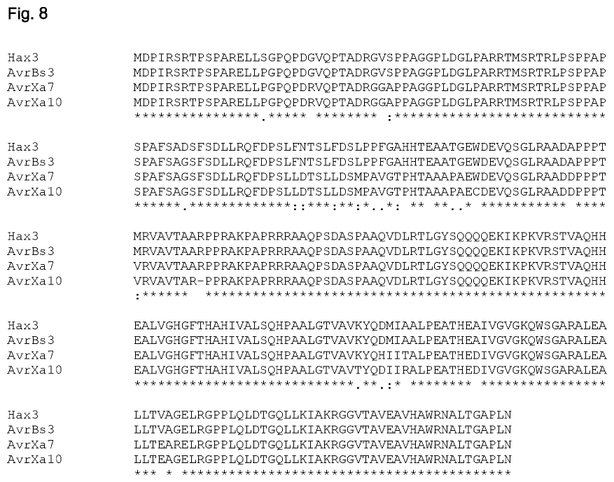

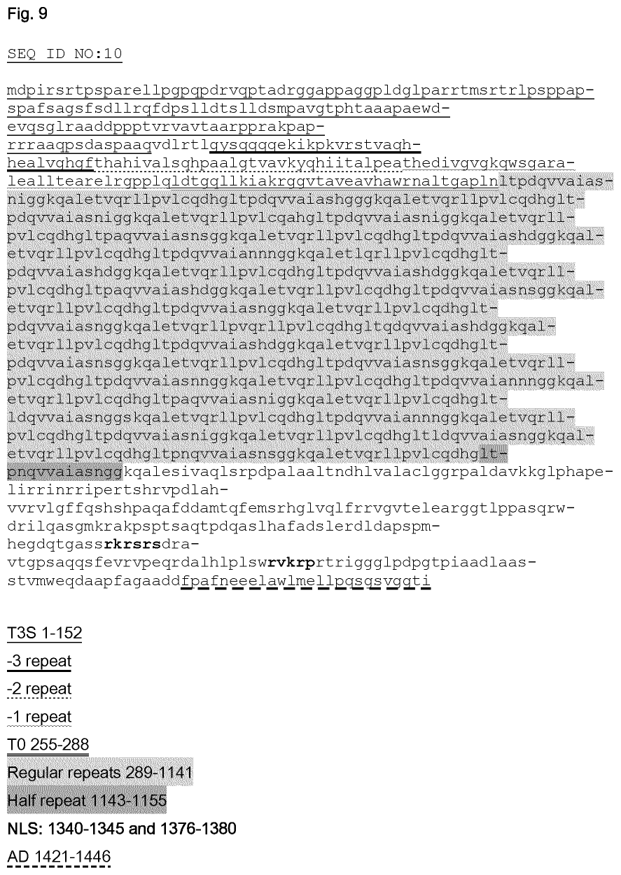

The NTRs of TALE polypeptides are known to be highly conserved. FIG. 8 shows an alignment of the NTRs of four different TALE polypeptides: AvrXa10 (SEQ ID NO: 18), AvrXa7 (SEQ ID NO: 19), AvrBS3 (SEQ ID NO: 20), and Hax3 (SEQ ID NO: 21). The N-terminal region of AvrXa7 is also shown in FIG. 9 (amino acids 1 to 288 of the sequence shown in FIG. 9). In an embodiment, the truncated TALE polypeptide comprises the NTR of AvrXa10 (or a variant thereof). In another embodiment, the truncated TALE polypeptide comprises the NTR of AvrBS3 (or a variant thereof). In another embodiment, the truncated TALE polypeptide comprises the NTR of AvrXa7 (or a variant thereof). In another embodiment, the truncated TALE polypeptide comprises the NTR of Hax3 (or a variant thereof). In an embodiment, the NTR of a TALE polypeptide comprises a sequence as shown FIG. 8, in particular a sequence shown in SEQ ID NO: 18, 19, 20 or 21, or is a variant thereof, wherein the amino acid sequence of the variant is, preferably, at least 50%, 60%, 70%, 80%, 85%, 90%, 92%, 95%, 97%, 98%, or 99% identical to the amino sequence shown in FIG. 8, in particular a sequence shown in SEQ ID NO: 18, 19, 20 or 21 (preferably, over the entire length of the aligned sequences).

In an embodiment, the NTR of a TALE polypeptide comprises amino acids 1 to 288 of SEQ ID NO: 10, or comprises an amino acid sequence which is at least 50%, 60%, 70%, 80%, 85%, 90%, 92%, 95%, 97%, 98%, or 99% identical to amino acids 1 to 288 of SEQ ID NO: 10.

In an embodiment, the truncated TALE polypeptide according to the present invention may further complete or incomplete Central Repeat Region (CRR) of a TALE polypeptide. Thus, the truncated TALE polypeptide may comprise the NTR and a complete CRR of a TALE polypeptide (preferably from N- to C-terminus). Alternatively, the truncated TALE polypeptide may comprise and an incomplete CRR of a TALE polypeptide (preferably from N- to C-terminus).

Preferably, a complete CRR comprises all amino acid tandem repeats of a full-length TALE polypeptide, whereas an incomplete CRR comprises not all amino acid tandem repeats of a full-length TALE polypeptide.

For example, an incomplete Central Repeat Region (CRR) comprises between 0.5 and 20 repeats (i.e. tandem repeats), such as between 0.5 and 10 repeats, between 0.5 repeats and 8 repeats, between 0.5 and 6 repeats, between 0.5 and 5 repeats, between 0.5 and 4 repeats, between 0.5 and 3 repeats, between 0.5 and 2 repeats, or between 0.5 and 1 repeats.

Also, it is envisaged that an incomplete CRR comprises less than 20 repeats or less than 10 repeats. In an embodiment, an incomplete CRR comprises less than 8 repeats. In another embodiment, an incomplete CRR comprises less than 5 repeats. In another embodiment, an incomplete CRR comprises less than 4 repeats. In another embodiment, an incomplete CRR comprises less than 3 repeats. In another embodiment, an incomplete CRR comprises less than 2 repeats. In another embodiment, an incomplete CRR comprises less than 1 repeat. Thus, an incomplete CRR may comprise only a incomplete repeat. For example, the incomplete Central Repeat Region comprises or consists of the amino acid sequence Itpeqvvaiasnsggkqal (SEQ ID NO:23).

A truncated TALE polypeptide comprising a complete DNA-binding domain is preferably obtained or obtainable by truncating a TALE polypeptide at the end of the DNA-binding domain of the TALE polypeptide. A truncated TALE polypeptide comprising an incomplete DNA-binding domain is preferably obtained or obtainable by truncating a TALE polypeptide within the DNA-binding domain of the TALE polypeptide. In other words, the truncated TALE polypeptide is the N-terminal portion of a TALE polypeptide which truncated within the DNA-binding domain of the TALE polypeptide (e.g. at positions described in the next paragraph).

In an embodiment, the TALE polypeptide is truncated within repeats the first twenty repeats, i.e. within any one of repeats 1 to 20, of the CRR of the TALE polypeptide. In a embodiment, the TALE polypeptide is truncated within repeats the first eight repeats, i.e. within any one of repeats 1 to 8, of the CRR of the TALE polypeptide. The repeat following the TO repeat is the first repeat. For example, the TALE peptide may be truncated within the first repeat of the DNA-binding domain, i.e. the CRR. Thus, the truncated TALE polypeptide comprises the NTR and a portion of the first repeat of the CRR of a TALE polypeptide. Further, the TALE peptide may be truncated within the second repeat of the DNA-binding domain (i.e. the repeat which follows the first repeat). Thus, the N-terminal portion comprises the NTR, the first repeat and a portion of the second repeat of a TALE polypeptide. Alternatively, the TALE polypeptide may be truncated between the second and third repeat of the DNA-binding domain. Thus, the N-terminal portion comprises the NTR, and the first and second repeat of a TALE polypeptide. Further, it is envisaged that the TALE polypeptide is truncated within the third repeat, between the third and fourth repeat, within the fourth repeat, between the fourth and fifth repeats, within the fifth repeat, between the fifth and sixth repeat, within the sixth repeat, between the sixth and seventh repeat, within the seventh repeat, between the seventh and eighth repeat, within the eighth repeat, between the eigth and ninth repeat, or within the ninth repeat of the DNA-binding domain of the TALE polypeptide. Further repeats may be present such as the 10^{th} repeat, 11^{th} repeat, the 12^{th} repeat etc. up to all repeats of the DNA-binding domain of the TALE polypeptide.

Thus, the TALE polypeptide can be truncated within repeats 1 to 20, repeats 1 to 15, repeats 1 to 10, repeats 1 to 8, repeats 1 to 6, repeats 1 to 5, repeats 1 to 4, repeats 1 to 3, repeats 1 to 2, or within repeat 1 of the CRR of the TALE polypeptide. Accordingly, the truncated TALE polypeptide can be obtained by is obtainable by truncating a TALE polypeptide at these positions.

The studies underlying the present invention suggest that the plant transformation could be improved even if the truncated TALE polypeptide does not have DNA-binding activity. In an embodiment of the present invention the truncated TALE polypeptide does not have DNA-binding activity. Nevertheless, it is also envisaged that the truncated TALE polypeptide has DNA-binding activity (e.g. if comprises a completed DNA-binding domain). Whether the truncated TALE polypeptide has DNA-binding activity or not can be assessed by the skilled person without further ado. Preferred assays for assessing whether a polypeptide has DNA-binding activity are e.g. disclosed in Zhang et. (2011) and Wan et al. (2016) which are both incorporated by reference.

In accordance with the present invention, the truncated TALE polypeptide shall comprise the N-terminal region of a TALE polypeptide, but shall lack certain parts of the TALE polypeptide (a i.e. a full length TALE polypeptide). Preferably, the truncated TALE polypeptide shall lack a transcriptional activation domain, in particular a TALE transcriptional activation domain. Accordingly, it shall not comprise a transcriptional activation domain, in particular a transcriptional activation domain of a TALE polypeptide. The term “transcriptional activation domain” (abbreviated “AD domain”) is well known in the art. For example, the AD domain of AvrXa7 spans amino acids 1421-1446 of SEQ ID NO: 10 (see FIG. 9).

In addition to the AD domain, the truncated TALE polypeptide may lack nuclear localization signals (NLSs). Accordingly, the truncated TALE polypeptide does not comprise nuclear localization signals (and an AD domain). AvrXa7 comprises NLS at positions 1340-1345 and 1376-1380 (see also FIG. 9).

In a preferred embodiment, the truncated TALE polypeptide lacks the entire CTR (C-terminal region). Preferably, the CTR is the region after the (downstream from) half repeat of the CRR. In a preferred embodiment, the truncated TALE polypeptide lacks the half repeat and the entire CTR (C-terminal region) of a full length polypeptide. Thus, the truncated TALE polypeptide may lack portions of the CRR (or even the complete CRR), and the entire CTR.

The truncated TALE polypeptide may comprise additional elements. In an embodiment, the truncated TALE polypeptide comprises a HA (human influenza hemagglutinin) tag at its N-Terminus, in particular a 3×HA tag, i.e. a triple HA tag. The HA tag is a fragment of the human influenza hemagglutinin polypeptide. Typically, a HA tag consists of the fragment corresponding to amino acids 98-106 of said polypeptide (YPYDVPDYA, SEQ ID NO: 11). A triple HA tag has preferably the sequence as shown in SEQ ID NO: 12 (YPYDVPDYAYPYDVPDYAYPYD-VPDYA). In an embodiment, said triple HA tag is encoded by a polynucleotide having a sequence as shown in SEQ ID NO: 13.

Although the truncated TALE polypeptide may comprise additional elements, it is envisaged that certain elements are not present. For example, the truncated TALE polypeptide preferably does not comprise a domain having nuclease activity (such as endonuclease activity). Accordingly, it is envisaged that the truncated TALE polypeptide as referred to herein does not have nuclease activity. Further, it is envisaged that the truncated TALE polypeptide does not have dioxygenase activity, methyltransferase activity, demethylase activity, transposase activity, and/or recombinase activity. Also, it is envisaged that the truncated TALE polypeptide is not fused to a fluorescent protein.

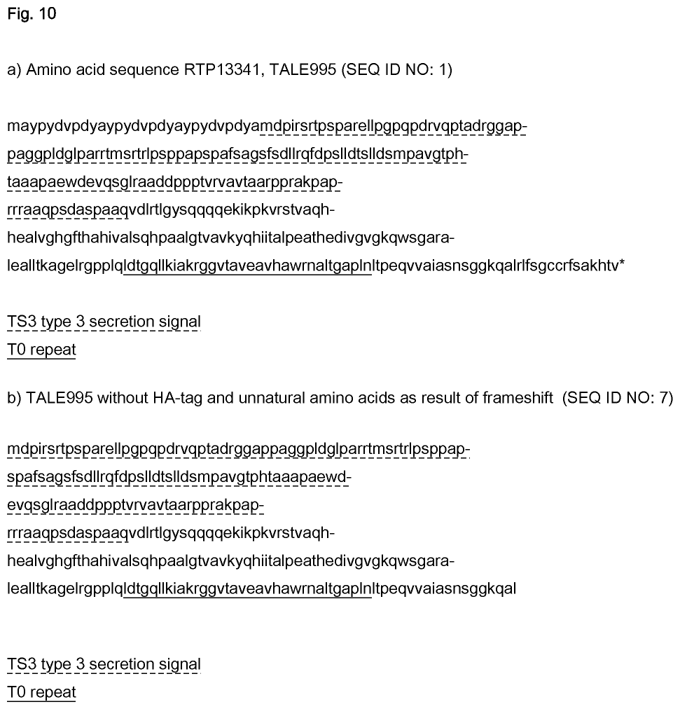

Preferred truncated TALE polypeptides comprise or consist of a sequence as shown in any one of SEQ ID NO: 1 to 3, or 7 to 9 (or are functional fragment thereof). The truncated TALE polypeptides having a sequence as shown in SEQ ID NO: 1, 2 and 3 were identified in the Examples underlying the present application. The truncated TALE polypeptides having a sequence as shown in SEQ ID NO: 7, 8 are 9 correspond to SEQ ID NO: 1, 2, and 3, but lack the 3×HA tag and the unnatural amino acids which result from a frameshift. The sequences are also shown in FIGS. 10 to 12.

The expression “truncated TALE polypeptide” as used herein encompasses also variants of the specific truncated TALE polypeptides as referred to herein (in particular, of truncated TALE polypeptides comprise or consist of a sequence as shown in any one of SEQ ID NO: 1, 2, 3, 7, 8, and 9). Such variants have at least the same or essentially the same biological activity as the specific truncated TALE polypeptides. In particular, a variant shall be capable of improving plant transformation as referred to herein. Also, a variant shall be capable of accelerating meristematic growth, when introduced into a plant (e.g. after introduction by Agrobacterium mediated transformation). Moreover, it is to be understood that a variant as referred to in accordance with the present invention shall have an amino acid sequence which differs from the specific polypeptides due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific truncated TALE polypeptides (preferably, over the entire length of said polypeptide).

In an embodiment of the present invention, the truncated TALE polypeptide, or functional variant thereof comprises or consists of:
(i) a sequence as shown in any one of SEQ ID Nos: 1, 2, 3, 7, 8, and 9, or a functional fragment thereof,
(ii) a sequence comprising amino acids 1 to 307 of SEQ ID NO: 10
(iii) a sequence comprising amino acids 1 to 443 of SEQ ID NO: 10
(iv) a sequence comprising amino acids 1 to 544 of SEQ ID NO: 10, or
(v) a sequence which is, in increasing order of preference, at least 50%, 60%, 70%, 80%, 85%, 90%, 95%, 98%, 99% or 100% identical to the sequence under (i), (ii), (iii) or (iv).

In an embodiment, the polynucleotide encoding said truncated TALE polypeptide, or variant thereof comprises or consists of
(i) a sequence as shown in any one of SEQ ID Nos: 4 to 6, or
(ii) a sequence which is, in increasing order of preference, at least 50%, 60%, 70%, 80%, 85%, 90%, 95%, 98%, 99% or 100% identical to the sequence under i).

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimize alignment between conserved motifs, as would be apparent to a person skilled in the art. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

In an embodiment, the algorithm of Needleman and Wunsch (see above) is used for the comparison of sequences. The algorithm is incorporated in the sequence alignment software packages GAP Version 10 and wNEEDLE. E.g., wNEEDLE reads two sequences to be aligned, and finds the optimum alignment along their entire length. When amino acid sequences are compared, a default Gap open penalty of 10, a Gap extend penalty of 0.5, and the EBLOSUM62 comparison matrix are used. When DNA sequences are compared using wNEEDLE, a Gap open penalty of 10, a Gap extend penalty of 0.5, and the EDNAFULL comparison matrix are used.

The expression “truncated TALE polypeptide” as used herein encompasses also functional fragments of the specific truncated TALE polypeptides, in particular, of truncated TALE polypeptides comprising or consist of a sequence as shown in any one of SEQ ID NO: 1, 2, 3, 7, 8, and 9.

In step b) of the method of the present invention at least one polynucleotide of interest is provided. The term “at least one” means one or more than one. Thus, it is envisaged to provide one, two, three, four, five or more polynucleotides of interest. Preferably, said polynucleotide(s) shall be stably introduced into the plant, i.e. it/they shall be introduced into the plant's genome. The expression “polynucleotide of interest” as used herein, preferably, refers to any polynucleotide that should be expressed the plant to be transformed, as described herein. In an embodiment, the polynucleotide of interest is a polynucleotide which encodes for a polypeptide (such as enzyme, a structural protein, or transcription factor). Further, the polynucleotide of interest may also encode for structural RNA or an active RNA such as an antisense RNA, an interfering RNA (such as a miRNA) or a ribozyme. Also, the at polynucleotide of interest may encode for a single-guide RNA (sgRNA).

In an embodiment of the methods of the present invention, at least two polynucleotides of interest are provided in step b) of the methods of the present invention (and are introduced into the plant cell in step c)). Preferably, the at least two polynucleotides of interest are different polynucleotides. One of the at least two polynucleotide of interest may be e.g. a marker gene which allows for the selection of plants which comprise said marker gene.

In an embodiment, said polynucleotide of interest is heterologous with respect to the plant. In another embodiment, said polynucleotide of interest is homologous with respect to the plant.

In a preferred embodiment of the method of the present invention, the polynucleotide of interest encodes for a gene-editing polypeptide. As used herein, the term “gene-editing polypeptide” refer to a polypeptide which is capable (either alone or in combination with other polypeptide) to edit the genome of the plant (to be transformed). Such gene-editing polypeptides are well known in the art and e.g. reviewed in (see Mohanta et al., 2017). In a preferred embodiment, the gene-editing polypeptide is a RNA-guided DNA endonuclease, in particular Cas9 (CRISPR associated protein 9). Also it is envisaged that said the gene-editing polypeptide is dCas9 (dead Cas9) which is a mutant form of Cas9 whose endonuclease activity is removed.

In a particularly preferred embodiment of the present invention, the polynucleotide of interest encodes a polypeptide selected from the group of polypeptides comprising Polydactyl zinc finger-FokI, Polydactyl zinc finger-cytidine deaminase, Polydactyl zinc finger-TevI, Polydactyl zinc finger-adenine deaminase, TALE-FokI, TALE-cytidine deaminase, TALE-TevI, TALE-adenine deaminase, CRISPR-Cas9, CRISPR-dCas9-FokI, CRISPR-dCas9-cytidine deaminase, CRISPR-dCas9-TevI, CRISPR-dCas9-adenine deaminase, CRISPR-Cpf1, CRISPR-dCpf1-FokI, CRISPR-dCpf1-cytidine deaminase, CRISPR-dCpf1-TevI, CRISPR-dCpf1-adenine deaminase, a homing endonucleases (such as I-SceI, I-CreI, I-CeuI), a CRE recombinase, and a fusion polypeptide of DNA binding domains to integrases and recombinases. Preferred polypeptides are CRISPR-Cas9 and TALE-FokI.

The polynucleotides provided in accordance with the method of the present invention, in particular the polynucleotide as referred to in step (a), item (ii) and/or the polynucleotide as referred to in step (b) of the above method, typically, are operably linked to a promoter. In particular, both the polynucleotide provided in step (a)(ii) and the polynucleotide provided in step (b) are operably linked to a promoter. Preferably, they are linked to different promoters.

Preferably, the promoter is heterologous with respect to the polynucleotide provided in step (a)(ii) and the polynucleotide provided in step (b), i.e. it is not the promoter which is naturally linked to said polynucleotides.

The term “operably linked” as used herein refers to a functional linkage between the promoter sequence and the polynucleotide encoding the truncated TALE polypeptide (or functional variant thereof) or the at least one at least one polynucleotide of interest. The term is to be understood as meaning, for example, the sequential arrangement of a regulatory element (e.g. a promoter) with a nucleic acid sequence to be expressed and, if appropriate, further regulatory elements (such as a terminator) in such a way that each of the regulatory elements can fulfil its intended function to allow, modify, facilitate or otherwise influence expression of said nucleic acid sequence. As a synonym the wording “operable linkage” or “operably linked” may be used. The expression may result, depending on the arrangement of the nucleic acid sequences, in sense or antisense RNA. Preferred arrangements are those in which the nucleic acid sequence to be expressed is recombinantly positioned behind the sequence acting as promoter, so that the two sequences are linked covalently to each other. The distance between the promoter sequence and the polynucleotide to be expressed is preferably less than 200 base pairs, especially preferably less than 100 base pairs, very especially preferably less than 50 base pairs.

The term “promoter” is well known in the art. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. It is to be understood that the promoter shall be active in the plant. Preferably, the promoter which is operably linked to the polynucleotide encoding the truncated TALE polypeptide (or variant thereof) is active in the transformed plant cells. Accordingly, it is envisaged that the truncated TALE polypeptide (or variant thereof) is expressed in the cells in which the polynucleotide has been introduced (in particular, in step c) of the methods of the present invention).

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

In an embodiment of the present invention, the promoter is a constitutive promoter. A “constitutive promoter” refers to a promoter that is transcriptionally active during most, but not necessarily all, phases of growth and development and under most environmental conditions, in at least one cell, tissue or organ. Examples for constitutive promoters are the actin promoter (McElroy et al, Plant Cell, 2: 163-171, 1990), CAMV 35S promoter (see e.g. Odell et al, Nature, 313: 810-812, 1985), the GOS2 promoter (See e.g. de Pater et al, Plant J November; 2(6):837-44, 1992, WO 2004/065596) or the ubiquitin promoter. In an embodiment, the constitutive promoter is the ubiquitin promoter, in particular the ubiquitin promoter from maize.

In an embodiment, the promoters is not the CAMV 35S promoter. E.g., the promoter is not the CAMV 35S promoter used for expressing the truncated TALE polypeptides shown in FIG. 1 of Schornack et al. (2004).

In another embodiment, the promoter is a developmentally-regulated promoter. A “develop-mentally-regulated promoter” is active during certain developmental stages or in parts of the plant that undergo developmental changes.

In another embodiment, the promoter is an inducible promoter. An “inducible promoter” has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to expo-sure to various pathogens.

In another embodiment, the promoter is an organ-specific or tissue-specific promoter. An “organ-specific” or “tissue-specific promoter” is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

In an embodiment, the organ-specific or tissue-specific promoter is selected from a root-specific promoter, a seed-specific promoter, an endosperm-specific promoter, an embryo specific promoter, an aleurone-specific promoter, a leaf specific promoter, and a meristem-specific promoter.

Further, the promoter may be a callus specific promoter. For example, the callus specific promoter may be the AoPR1 promoter (as disclosed in U.S. Pat. No. 6,031,151), the 1178-21 promoter (as disclosed in WO 02/097085), the CSP-promoter (as disclosed in Wakasa et al., 2007), or the MsPRP2 promoter (as disclosed in Winicov et al., 2004).

Typically, the polynucleotide provided in step (a)(ii) and the polynucleotide provided in step (b) are also operably linked to a terminator. The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

In step (c) of the aforementioned method, the polypeptide or variant thereof provided in step (a), item (i), or the polynucleotide, or variant thereof, provided in step (a), item (ii), and the at least one polynucleotide of interest provided in step (b) are introduced into the plant, i.e. a plant cell. Preferably, the polynucleotide, or variant thereof, provided in step (a), item (ii), and the at least one polynucleotide of interest provided in step (b) are introduced into the plant, e.g. by Agrobacterium mediated plant transformation. Typically, the polynucleotides are introduced simultaneously in the plant.

The combined introduction of the polypeptide or variant thereof provided in step (a), item (i) and the at least one polynucleotide of interest provided in step (b), or the combined introduction of the polynucleotide or variant thereof provided in step (a), item (ii) and the at least one polynucleotide of interest provided in step (b) allows for improving plant transformation.

Plants that are particularly useful accordance with the present invention (and thus plants that are be transformed) include all plants which belong to the superfamily Viridiplantae, in particular plants selected from the list comprising Acer spp., Actinidia spp., Abelmoschus spp., Agave sisalana, Agropyron spp., Agrostis stolonifera, Allium spp., Amaranthus spp., Ammophila arenarla, Ananas comosus, Annona spp., Aplum graveolens, Arachls spp, Artocarpus spp., Asparagus officinaliS, Avena spp. (e.g. Avena satlva, Avena fatua, Avena byzantine, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinoses, Camellia sinensis, Cannaindica, Cannabis sativa, Capsicum spp., Carex elate, Carica papaya, Carissa macrocarpa, Carya spp., Carthamus tinctorius, Castanea spp., Celba pentandra, Cichorium endivia, Cinnamomum spp., Citrullus lanatus, Citrus spp., Cocos spp., Coffee spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Eleeis (e.g. Elaeis guineensis, Elaeid oleiferal, Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus thus annuus), HemerocalliS fulva, Hibiscus spp., Hordeum spp. (e.g. Hordeum vulgare), Ipomoea batatas, Juglans spp., Lactuca sativa, Lathyrus spp., Lens culinais, Linum usitatissimum, Litchi chinensis, Lotus spp., Luffa acutangula, Lupinus spp., Luzula sylvatica, Lycoperiscon spp. (e.g. Lycopersicon esculentum, Lycopersicon lycopersicum, Lycopersicon pynforme), Macrotyloma spp., Malus spp., Malpighia emarginata, Mammea americana, Mangifera indica, Manihot spp., Manlikara zapota, Medicago sativa, Melllotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalanis arundinacea, Phaseolus spp., Phleum pretense, Phoenb (spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereals, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Tnfolium spp., Tripsacum dactyloides, Tnticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

In an embodiment, the plant is a monocotyledonous plant (such as maize or rice which were transformed in the studies underlying the present invention, see Examples section). In another embodiment, the plant is a dicotyledonous plant (such as sunflower or Arabidopsis thaliana).

According to an embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, and potato. According to another embodiment of the present invention, the plant is a cereal. Preferred cereal include rice, maize, wheat, barley, millet, rye, triticale, sorghum, emmer, spelt, einkorn, teff, milo and oats.

In an embodiment of the present invention, the plant is not a tobacco plant. In another embodiment, the polypeptide and/polynucleotide provided in steps a) i) and ii) are not introduced into a plant cell by leaf infiltration of tobacco leafs.

The method of the present invention shall allow for improving plant transformation, in particular transformation of the at least one polynucleotide of interest as referred to herein into a plant. The transformation of said at least one polynucleotide is interest into a plant is improved by the co-introduction of the polypeptide provided in step (a)(i) and/or the polynucleotide provided in step (a)(ii) of the method of the present invention (see step c). Preferably, plant transformation is improved as compared to plant transformation which does not comprise the introduction of the polypeptide provided in step (a)(i) and/or the polynucleotide provided in step (a)(ii).

The term “transformation” as referred to herein encompasses the transfer of a polynucleotide, herein referred to a polynucleotide of interest, into a plant host cell. Plant tissue capable of subsequent clonal propagation, whether by organogenesis or embryogenesis, may be transformed with a genetic and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotides as referred to herein are preferably transiently and more preferably stably introduced into a host cell. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. In a preferred embodiment of the method of the present invention, the term “plant transformation” refers to Agrobacterium-mediated plant transformation. Thus, the at least the at least one polynucleotide of interest as provided in step b) of the method of the present invention is introduced into the plant by Agrobacterium-mediated plant transformation. In a preferred embodiment, both the polynucleotide provided in step (a)(i) and the at least one polynucleotide provided in step (b) are introduced, in particular stably introduced, into the plant by Agrobacterium-mediated plant transformation. In order to allow for the introduction into the plant, the polynucleotide encoding said truncated TALE polypeptide, or functional variant thereof, as provided in step (a)(ii), and the at least one polynucleotide of interest as provided in step b) are present in the same T-DNA or in different T-DNAs. Accordingly, the present invention also contemplates a T-DNA comprising the polynucleotide as defined in step (a)(ii) and at least one polynucleotide of interest as defined in step b) of the method of the present invention. Further, the present invention also contemplates a set of two T-DNAs, wherein the first T-DNA comprises the polynucleotide as defined in step (a)(ii) and the second T-DNA comprises at least one polynucleotide of interest as defined in step b) of the method of the present invention.

The two polynucleotides as referred to in the previous paragraph can be introduced simultaneously into the plant in step c) of the methods of the present invention. E.g., they may be co-transformed into a plant cell by Agrobacterium mediated transformation.

Alternatively, the two polynucleotides can be introduced into the plant at different time points. For example, in a first step the polypeptide or polynucleotide provided in step (a) is introduced into the plant, followed by the introduction of the at least one polynucleotide of interest provided in step (b) in a second step. In an embodiment, the plant into which the polynucleotide of interest of step (b) is introduced may already stably comprise the polynucleotide encoding a truncated transcription activator-like effector (TALE) polypeptide. In this case, step (c) of the aforementioned method comprises the introducing the at least one polynucleotide of interest provided in step (b) into a plant which comprises (and expresses) the polynucleotide as defined in step (a)(ii).

The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38. Methods for maize transformation, are e.g. described in either Ishida et al. (Nat. Biotechnol 14(6): 745-50, 1996) or Frame et al. (Plant Physiol 129(1): 13-22, 2002). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis(Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media.

The transformation method to be applied in accordance with the present invention, preferably, requires formation of a callus. In an embodiment, the transformation method is not germline line transformation (such as the Floral Dip transformation method). Further, it is envisaged that the transformed explants are not germinating seeds.

Transformation methods may also include the use of liposomes, electroporation, chemicals that increase free DNA uptake, injection of the DNA directly into the plant, particle gun bombardment, transformation using viruses or pollen and microinjection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like.

It is to be understood that the present invention not limited to the stable introduction of the polynucleotides as referred to herein. Rather, the polypeptide and the polynucleotide(s) as referred to in step (c), in particular the truncated TALE polypeptide, or the polynucleotide encoding said truncated TALE polypeptide, can be also transiently introduced into the plant (in step (c) of the method of the present invention. E.g., transient introduction can be obtained by particle bombardment, by cell penetrating peptides, by using suitable viral vectors, or by using zwitterions, by using PEG, by using nanoparticles, by using whiskers or by using type 3 or type 4 secretion systems. transiently; either as episomal-encoded DNA (including viral vectors) or as mRNA/protein. For example, Agrobacterium may be used to transiently introduce the truncated TALE polypeptide as a fusion with the type IV secretion signal of VirF. In principle, a type III secretion system could be also used to introduce the truncated TALE polypeptide. Further, fusions of the truncated TALE polypeptide to VirD2 or VirE2 are envisaged.

The present invention also contemplates a combination of stable introduction and transient introduction. For example, it is contemplated that the at least one polynucleotide of interest as provided in step b) is stably introduced into the plant by Agrobacterium-mediated transformation, whereas the polypeptide provided in step (a)(i) or the polynucleotide provided in step (a)(ii) of the method of the present invention is transiently introduced into the plant. In this case, it is not required to remove the polynucleotide encoding for the truncated TALE polypeptide at later stages.

The plant cells transformed by the method of the present invention can be regenerated via all methods with which the skilled worker is familiar. Generally, after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. Advantageously, the T1 plants generated by the method of the plants did not show the phenotype of the TO plants. This allows a better analysis of the phenotype caused by the presence of the polynucleotide of interest.

As set forth above, the introduction of the polypeptide or polynucleotide provided in step (a) of the methods of the present invention allows for an improvement of plant transformation. The expression “improvement of plant transformation” as used herein typically refers to an improved transformation of the at least one polynucleotide of interest into a plant. As set forth above, plant transformation is improved as compared to plant transformation without the introduction of the polypeptide or polynucleotide provided in step a), i.e. as compared to a method which does not comprise, i.e. lacks, the step of introducing said polynucleotide or polypeptide.

In an embodiment, the improvement of plant transformation is selected from
(i) enhanced transformation efficacy,
(ii) a faster growth of transformed calli,
(iii) a faster generation of TO plants, and
(iv) an increased biomass of generated TO plants.

The terms “increased” or “enhanced” in the context of a plant transformation are inter-changeable and shall mean in the sense of the application an increase of at least 10%, preferably at least 15% or 20%, more preferably 25%, 30%, 35% or 40%, even more preferably at least 70% or most preferably at least 100% of the transformation efficacy, the diameter of generated calli, or the biomass, in particular in comparison a control transformation which lacks the step of introducing the polynucleotide or polypeptide as defined in step a) of the method of the present invention.

A faster generation of TO plants means that the regeneration of TO requires less time when carrying out the method of the present invention in comparison a control transformation which lacks the step of introducing the polynucleotide or polypeptide as defined in step a) of the method of the present invention. Preferably, the regeneration of TO plants may require at least 10%, more preferably at least 15%, and most preferably at least 20% less time than the control. For example, it has been shown in the studies underlying the present invention that rice plants produced by the present method, regenerated 1-2 weeks earlier than control plants. Thus, the time needed for the regeneration of TO plants may be shortened by about one to two weeks by the method of the present invention.

Further, a faster growth of transformed calli was observed. The transformed calli obtained by the method of the present invention had an increased diameter as compared to calli that were obtained by a control transformation.

Step (d) of the aforementioned method for generating a transgenic plant comprises the regeneration of a transgenic plant comprising the at least one polynucleotide of interest. Thus, a whole plant is regenerated from a plant cell which comprises the at least one polynucleotide of interest. Said regenerated (whole) plant shall comprise, preferably stably comprise the at least one polynucleotide of interest. Preferably, the regenerated plant further comprises (in particular stably comprises) the polynucleotide encoding the truncated TALE polypeptide (in case the polynucleotide as forth in step (a), item (ii) has been introduced).

After introduction of the polynucleotide in step b) and optionally the polynucleotide as set forth in step a)(ii) into the plant, in particular after transformation target cells, cell division is induced by specific plant hormones in order to grow a callus from a transformed plant cell. After callus induction, the resulting callus is transferred to a medium allowing shoot induction. The callus is incubated (under in vitro conditions) on said medium until shoots are formed. After shoot formation, the shoot is transferred to a medium that allows for root formation (under in vitro conditions). After root formation, regenerated plantlets (i.e. shoots with roots) are usually transferred from in vitro conditions to ex vitro conditions, mostly to soil or hydroponic media under greenhouse conditions.

The definitions and explanations given herein above in connection with the methods of the present invention preferably apply mutatis mutandis to the following.

The present invention further concerns a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete TALE Central Repeat Region (CRR), and wherein said truncated TALE polypeptide does not comprise a transcriptional activation domain (as defined elsewhere herein in connection with the method of the present invention).

In an embodiment of the present invention, said polypeptide consists of or comprises
(i) a sequence as shown in any one of SEQ ID Nos: 1, 2, 3, 7, 8, and 9, or a functional fragment thereof or
(ii) a sequence which is, in increasing order of preference, at least 50%, 60%, 70%, 80%, 85%, 90%, 95%, 98%, 99% or 100% identical to the sequence under i).

Moreover, the present invention relates to a polynucleotide encoding for the truncated TALE polypeptide of the present invention.

In an embodiment, said polynucleotide consists of or comprises
(i) a sequence as shown in any one of SEQ ID Nos: 4 to 6, or
(ii) a sequence which is, in increasing order of preference, at least 50%, 60%, 70%, 80%, 85%, 90%, 95%, 98%, 99% or 100% identical to the sequence under i).

Further the present invention concerns a vector comprising the polynucleotide of the present invention. Preferably, said vector is an expression vector, i.e. a vector with allows for expression of the truncated polypeptide of the present invention. Thus, it is envisaged that said polynucleotide is operably linked to a promoter. In an embodiment, said promoter is heterologous with respect the polynucleotide of the present invention.

The present invention also concerns a host cell, in particular a non-human host cell, comprising the polypeptide of the present invention, the polynucleotide of the present invention and/or the vector of the present invention.

In preferred embodiment, said host cell is a plant cell, e.g. cell from a plant as specified above. For example, the host cell may be a cell from a monocotyledonous plant (such as a maize or rice cell) or a dicotyledonous plant (such as a sunflower or soybean cell). In another preferred embodiment, the host cell is a bacterial cell. For example, the bacterial cell is an E. coli cell or an Agrobacterium cell such as an Agrobacterium tumefaciens cell.

The present invention also concerns a plant comprising the polypeptide of the present invention, the polynucleotide of the present invention and/or the vector of the present invention.

In a preferred embodiment of the aforementioned host cell or plant, the host cell or plant preferably further comprises at least one polynucleotide of interest. The polynucleotide of interest has been defined elsewhere herein. The definition applies accordingly.

Further, it is envisaged that the host cell or plant stably comprises the polynucleotide encoding for the truncated TALE polypeptide of the present invention.

Also the present invention contemplates the use of the truncated TALE polypeptide of the present invention, the polynucleotide of the present invention, or the vector of the present invention for improving plant transformation. Further, the present invention contemplates the use of the truncated TALE polypeptide of the present invention, the polynucleotide of the present invention, or the vector of the present invention for generating a transgenic plant comprising at least one polynucleotide of interest.

Finally, the present invention relates to a kit comprising the polypeptide of the present invention, the polynucleotide the of the present invention, or the vector (or the expression vector of the present invention). In a preferred embodiment, the kit further comprises at least one polynucleotide of interest (as defined elsewhere herein).

The Figures show:

FIG. 1: Overview of the constructs used in the studies underlying the present invention (sequences in the figures: TNACGCGGGAN is SEQ ID NO: 15, TNATCTGNA is SEQ ID NO: 16)

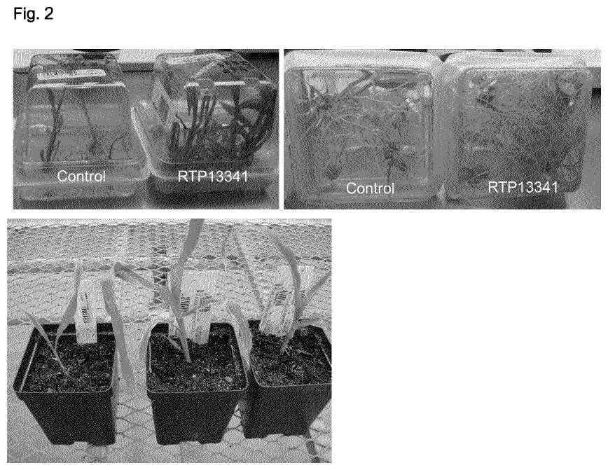

FIG. 2: RTP13341 promotes transformation efficacy, boosts regeneration and shortens time lines in the generation of TO plants. The control (RTP11885) and RTP13341 plants were derived from the same transformation date and an equal number of plants are present in the boxes.

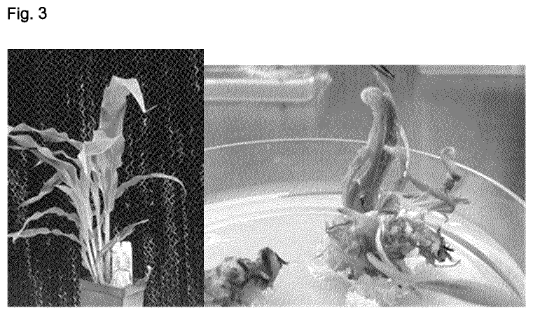

FIG. 3: Tillering (left, RTP13336) and abnormal leaf development (right, RTP13346)

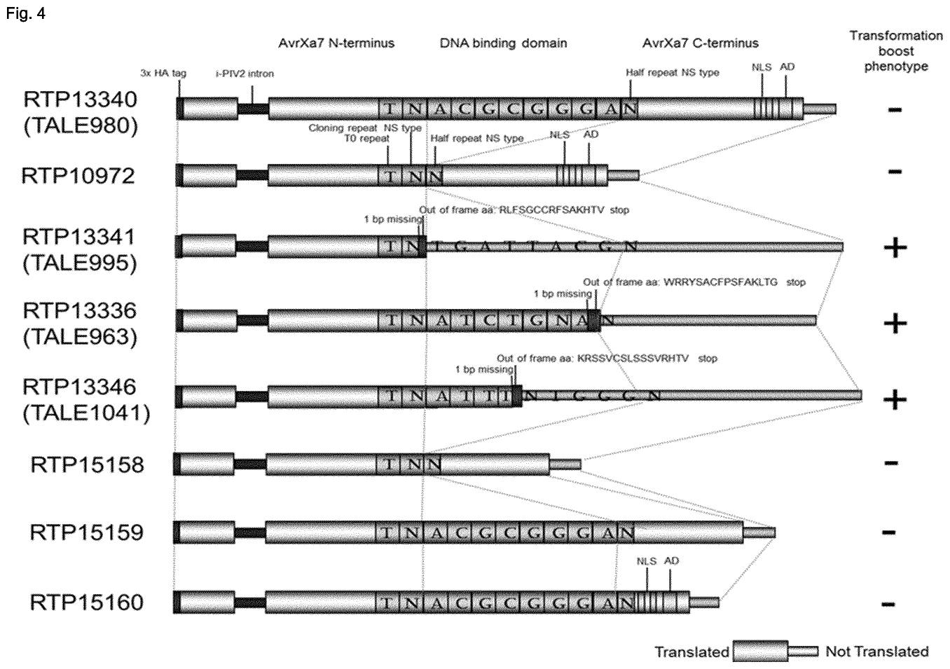

FIG. 4: Further constructs used in the present studies

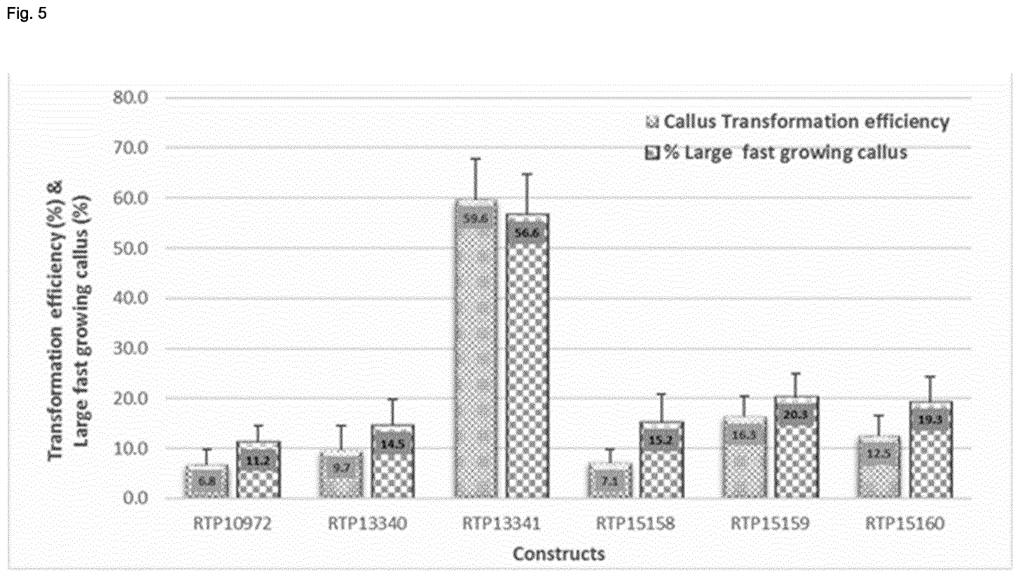

FIG. 5. Transformation efficiency and % large fast-growing callus following transformation using six different TALE constructs (including control construct) in corn.

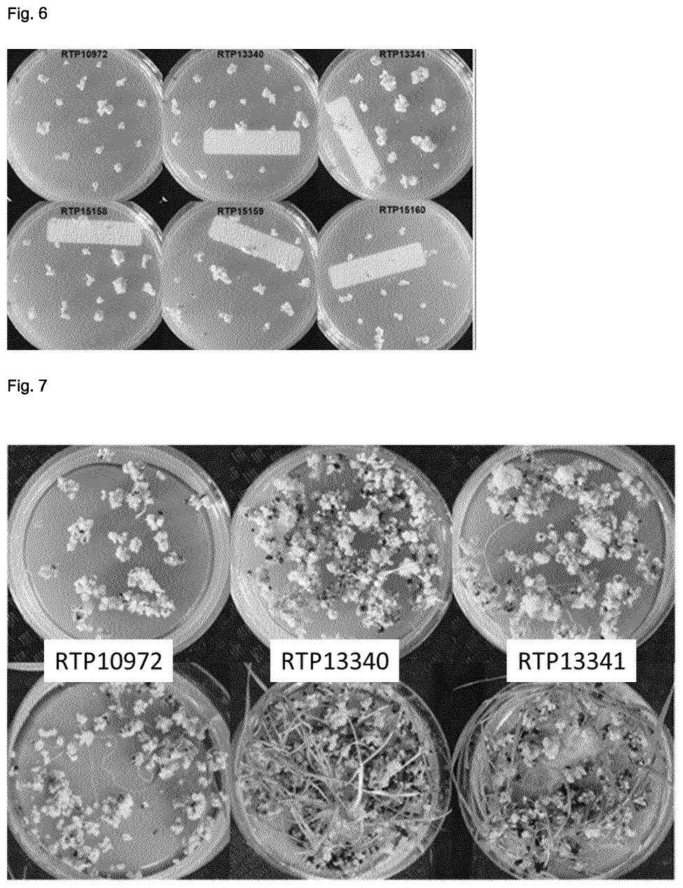

FIG. 6. Corn Transformation using six TALE constructs (including control construct). Callus growth on culture media

FIG. 7. Rice transformation using three TALE constructs (including control construct). Upper panel—DsRed expressing callus from three constructs and, Lower panel—Plant regeneration from DsRed expressing callus from three constructs.

FIG. 8. Alignment between four N-terminal regions of different TALE polypeptides. As it can be seen from the alignment, there is a high degree of sequence identity between TALE polypeptides from different species. (AvrXa10: SEQ ID NO: 18, AvrXa7: SEQ ID NO: 19, AvrBS3: SEQ ID NO: 20, Hax3: SEQ ID NO: 21). The N-terminal region comprises the T3S and translocation signal and repeats −3, −2, −1 and 0 of AvrXa10, AvrXa7, AvrBS3, and Hax3, respectively.

FIG. 9 Domains in AvrXa 7 (SEQ ID NO: 10)

FIG. 10 a) amino acid sequence RTP13341 (TALE995). The T3S and translocation signal and the TO repeat are underlined.
b) amino acid sequence RTP13341 (TALE995) without HA-tag and unnatural amino acids as result of frameshift

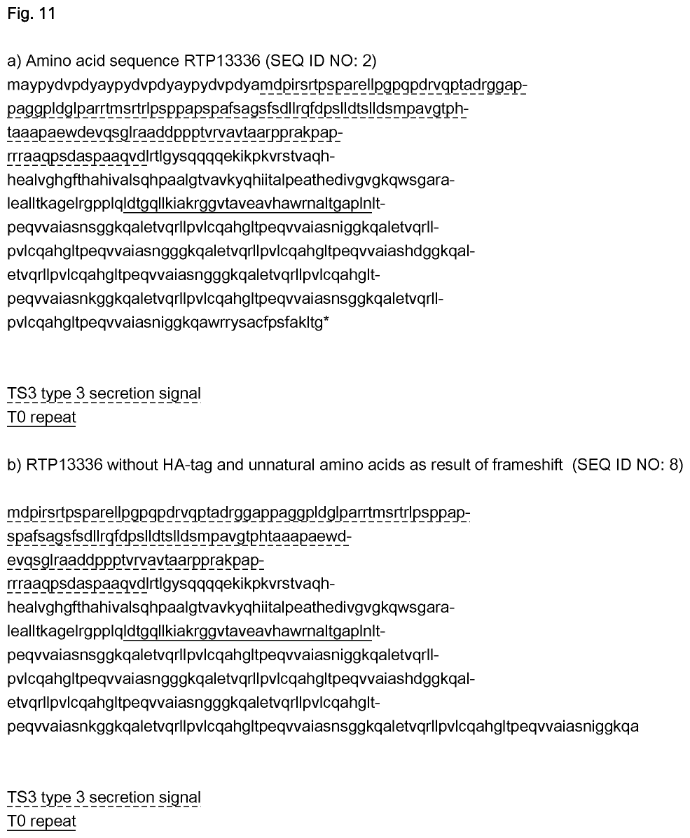

FIG. 11 a) amino acid sequence RTP13336. The T3S and translocation signal and the TO repeat are underlined.
b) amino acid sequence RTP13336 without HA-tag and unnatural amino acids as result of frameshift

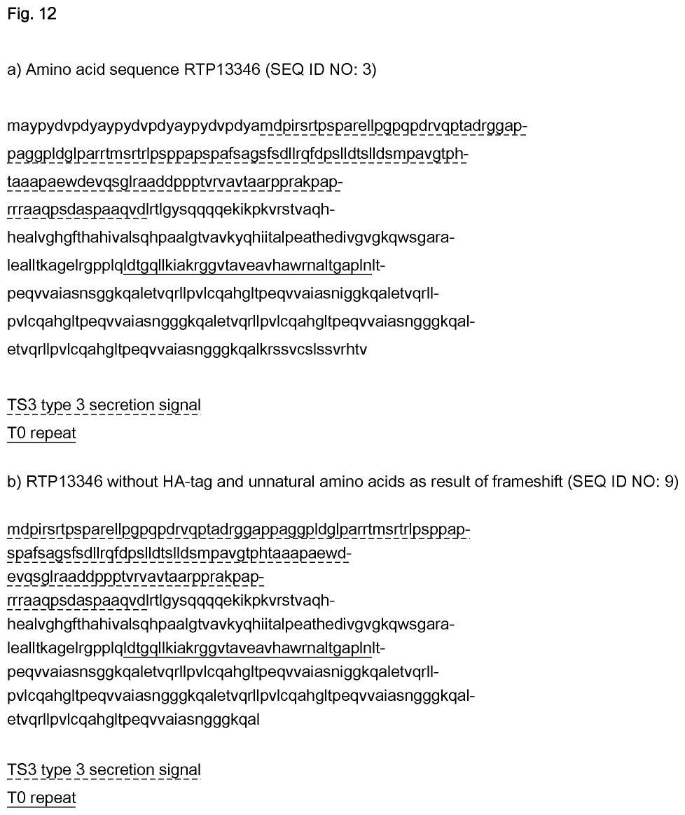

FIG. 12 a) amino acid sequence RTP13346. The T3S and translocation signal and the TO repeat are underlined.
b) amino acid sequence RTP13346 without HA-tag and unnatural amino acids as result of frameshift

### Examples

Identification of a Truncated TAL Effector (RTP13341) which Boosts Transformation, Sequence and Phenotype

RTP13341 containing TALE995 under control of the constitutive ZmUBI promoter was isolated after high throughput cloning of randomized DNA concatemers encoding TALE DNA binding repeats in the plant transformation scaffold vector RTP10972 (FIG. 1). Repeats were concatemerized by ligating CCCT 5′ overhangs to GGGA 5′ overhangs of individual, double stranded TALE repeat-encoding DNA modules and a chain of 8 repeats intended to recognize the sequence TGATTACG was cloned into a BsmBI site in the cloning repeat of RTP10972 using compatible cohesive ends (FIG. 1). The first cloned repeat, which has the RVD sequence for recognition of T, possessed a single base pair deletion immediately following CCCT (FIG. 1). This results in a frame-shift which adds the sequence RLFSGCCRFSAKHTV (SEQ ID NO: 14) to the truncated TALE before encountering a stop codon. All remaining TALE DNA binding repeats and the C-terminus of TALE including NLSs and AD are not translated. A TALE requires the presence of at least 8 units in the DNA binding domain to meet the minimal affinity for transcriptional activation (Boch et al., 2009) and amino acids after the half repeat are required for proper folding of the DNA binding domain (Zhang et al., 2011). RTP13341 was transformed into corn model line HillaxA188/J553 using Agrobacterium and immature embryos as target tissue for T-DNA delivery. Transformation efficacy was boosted by a factor 2 fold over transformation of controls which lacked the TALE portion

TABLE 1
Qualitative data on transformation and growth changes observed between
RTP13341 and control constructs in multiple experiments.

| | RTP11885 | RTP10927 | RTP13341 |
|---|---|---|---|
| Transient Expression | 100's | 100's | 100's |
| (DsRED cells per embryo) | | | |
| % Stable Transformation Efficiency | 50% | 50% | 100% |
| (unique events/embryos started) | | | |
| Callus event size diameter in cm | 1-2 | 1-2 | 3-5 |
| (3 weeks on selection) | | | |
| Weeks from Transformation to | 6 | 6 | 3 |
| plantlet formation | | | |
| Overall biomass or size | normal | normal | larger |
| Weeks to anthesis | 6-8 | 6-8 | 4-6 |
| (from transplantation greenhouse) | | | |
| seed number | normal | normal | slightly |
| (qualitative, more data needed) | | | larger |

of the T-DNA (RTP11885, see appendix) and RTP10972. Most plantlets developed significantly faster (FIG. 2) allowing transfer to the greenhouse 3 weeks earlier. For further confirmation, the transformation experiment was repeated both for model line and J553 inbred line. In both cases the same results were found. Both the empty scaffold vector RTP10972 as well as regular TALEs, for instance TALE980 in RTP13340 (also derived from RTP10972), did not yield the growth-promoting phenotype. The empty scaffold TALE, like TALE995 has very low affinity to bind DNA. Therefore, the absence of the C-terminus of TALE is a prerequisite for obtaining the reported phenotype. TALE995 does not improve Agrobacterium function, because of the presence of the i-PIV2 intron in the 5′ coding end. We determined the T-DNA integration copy number and observed no abnormalities as compared to transformation of controls (Table 2). We identified two additional constructs from our libraries which are not encoding a fully functional TALE. RTP13336 (FIG. 1 and Table 1) encodes a truncated polypeptide that ends after the cloned DNA binding units ATCTGNA and RTP13346 ends after the cloned binding units ATTT (not shown). Both are not expected to be able to bind DNA and do not possess translated NLS and AD. Phenotypes were shown to be the same and confirmed the RTP13341 result.

TABLE 2
Copy numbers determined through TaqMan assays on t-nos.

| Copy # | 1 | 2 | 3 | 4 |
|---|---|---|---|---|
| RTP13341 | 8 (47%) | 5 (29%) | 1 (6%) | 3 (18%) |
| RTP13336 | 6 (38%) | 3 (19%) | 2 (12%) | 5 (31%) |

Approximately 85% of all events with RTP13341, RTP13336 and RTP13346 displayed the enhanced transformation, regeneration and growth phenotype. In more rare cases we observed excessive tillering or abnormal leaf development (FIG. 3). All phenotypes seem to be linked and may represent different manifestations of increased meristematic activity. Surprisingly, in the

Further Constructs

A new set of experiments was initiated to answer two basic questions: (1) what are the exact prerequisites for truncated TALE mode of action in corn and (2) does TALE995 also work in other crops. Rice was selected since the action in maize is not dependent on specific DNA binding.

We constructed plasmids RTP15158 (RTP10972 without NLS and AD), RTP15159 (RTP13340 without NLS and AD) and RTP15160 (RTP13340 without the C-terminus FIG. 4) for the first goal and introduced RTP13341 into rice cv Nipponbare for the second goal.

Corn Transformation Using TALE Vectors
Transformation of corn (cv. J553) were performed using six constructs—RTP10972, RTP13340, RTP13341, RTP15158, RTP15159, RTP15160
Transformation replicated 9 times, for each replication immature embryos from one corn ear were divided among all the six constructs
Transformed embryos were placed on AHAS selection for initially 4 weeks, and then transferred to fresh AHAS selection media for further 4 weeks
After 2 weeks of transformation, fast growing embryo-deriving callus were counted based on their size (>˜4 mm)
For transformation efficiency, DsRed expressing embryo-derived callus (callus with at least one foci) were counted after 4 weeks of selection. These data were collected from all the 9 replications.
To study developmental time line of corn callus, all the callus within the treatment were visually ranked according to their appearance, which are: 0=Non-regenerable, 1=potential embryogenic, 2=Embryogenic structures Type-2 callus, 3=Organized Type-1 callus, 4=Callus with photosynthetic shoots/plantlets. These data were collected from 6 replications.

TABLE 3
Physiological characteristics of corn callus after 7-8 weeks of selection.
All the callus were visually ranked according to their appearance following
transformation and selection. Data shows % of callus with SEm.

| | Physiological characteristics of callus after 7-8 weeks of selection | | | | |
|---|---|---|---|---|---|
| | | % Type-1 | | | |
| | % Callus with | callus, with | % Embryogenic | | |
| | photosynthetic | green | Structure - | % Potential | % Non |
| Constructs | Shoot/Plantlets | structure | Type-2 | embryogenic | regenerable |
| RTP10972 | 2.5 ± 1.3 | 4.7 ± 1.8 | 6.8 ± 1.0 | 14.8 ± 2.1 | 71.2 ± 3.7 |
| RTP13340 | 3.1 ± 1.9 | 5.9 ± 2.1 | 3.9 ± 03 | 16.8 ± 2.3 | 70.3 ± 3.5 |
| RTP13341 | 28.7 ± 4.0 | 23.1 ± 3.3 | 12.0 ± 2.6 | 10.8 ± 2.3 | 25.4 ± 5.1 |
| RTP15158 | 2.4 ± 1.0 | 3.0 ± 1.1 | 10.4 ± 3.0 | 19.1 ± 4.2 | 65.1 ± 6.2 |
| RTP15159 | 4.8 ± 2.0 | 7.8 ± 3.0 | 10.8 ± 2.0 | 14.4 ± 1.5 | 62.2 ± 5.3 |
| RTP15160 | 3.0 ± 1.4 | 3.1 ± 1.1 | 7.2 ± 1.6 | 19.4 ± 2.8 | 67.3 ± 3.9 |

Results
Transformation efficiency from construct carrying truncated TALE (RTP13341) is ˜60% which is 10× higher than control construct (RTP10972), and 4-6× higher than other TALE constructs (FIG. 5)
High percentage of callus which are transformed with truncated TALE (construct RTP13341), grows significantly faster and larger than callus transformed with other constructs (FIG. 5)
Multiple events expressing DsRed were observed in most of the embryo-derived callus transformed with RTP13341 constructs, whereas only 1 or 2 events expressing DsRed were observed in callus transformed with other constructs (not shown)
Around 29% of callus transformed with RTP13341 formed photosynthetic shoot/plantlets compared to 2-5% of callus transformed with other constructs (Table 3)
Callus transformed with truncated TALE (RTP13341) had 3-7× higher percentage of regenerable, Type-1 callus compared to callus transformed with control and other TALE constructs (Table 3)
Only 25% of callus transformed with RTP13341 were non-regenerable after 7-8 weeks of selection whereas 65-70% of callus transformed with other constructs were non-regenerable (Table 3). These suggests shorter developmental timeline in in-vitro for callus expressing TALE995 construct RTP13341.

Rice Transformation Using TALE Vectors
Transformation of rice (cv. Nipponbare) were performed using three constructs—RTP10972, RTP13340, RTP13341
Transformation performed only once
Transformed germinating seeds were placed on AHAS selection for initially 3-4 weeks followed by micro-calli isolations and then transferred to fresh AHAS selection media for further 3-4 weeks
For callus transformation efficiency, DsRed expressing callus (callus with at least one foci) were counted after 6 weeks of selection.
After 7-8 weeks of selection, callus events were transferred to regeneration media and regenerated plant events expressing DsRed in roots were counted for plant transformation efficiency

TABLE 4
Number of callus and plant events expressing DsRed following
transformation using three different TALE constructs
(including control construct) in rice. Percentage in
parenthesis are transformation efficiencies.

| | | | # DsRed | | | |
|---|---|---|---|---|---|---|
| | | | Expressing | | # Transgenic | |
| | Explant | # Isolated | Callus events | | plant events | |
| Constructs | used | micro -calli | (% efficiency) | | (% Efficiency) | |
| RTP10972 | 23 | 40 | 7 | (30.4%) | 2 | (8.7%) |
| RTP13340 | 11 | 46 | 19 | (172.7%) | 5 | (45.5%) |
| RTP13341 | 25 | 163 | 142 | (568%) | 21 | (84%) |

Callus transformation efficiency from construct carrying truncated TALE (RTP13341) is >500% which is ˜19× higher than control construct (RTP10972), and 3× higher than another TALE RTP13340 construct (Table 4)
Plant transformation efficiency from construct carrying truncated TALE (RTP13341) is 84% which is ˜10× higher than control construct (RTP10972), and double than another TALE construct RTP13340 (Table 4).

Summary/Conclusions

We describe a general increase in transformation efficacy in corn by overexpression of the N-terminus of the transcription activator-like effector (TALE) AvrXa7 from Xanthomonas oryzae in the plant transformation vector RTP13341. The transformation efficacy was increased by a factor of more than 2 fold over controls and plants were moved to the greenhouse 3 weeks earlier. The effect is independent of specific DNA binding and transcriptional activation as both corresponding domains are not functionally present in the truncated protein. This notion was further supported by the fact that introduction of RTP13341 into rice produced similar results. RTP13341 was fortuitously isolated from a large collection of plasmids with randomized TALEs. The randomization is dependent on PCR and this step is most likely responsible for a single base pair deletion in the first 34 aa repeat after the TO repeat, which disrupts the open reading frame and leads to a stop 15 amino acids downstream from the deletion. The absence of the transcriptional activation domain (AD), nuclear localization signal (NLS) and C-terminus is a prerequisite for the positive effect of RTP13341. The positive effect was confirmed in maize model line J553x(HillaxA188) and J553 and rice cv. Nipponbare. The presence of an intron implies that improved Agrobacterium fitness, plant cell attachment and T-DNA transfer are not the leading cause of improved transformation efficacy. Copy numbers were found to be normal. Thus, RTP13341 seems to have a general effect on plant development giving more cells with T-DNA integration events the opportunity to regenerate while accelerating development at the same time. This truncated TALE polypeptide, if applied may have value in genome editing, allowing cells with rare genome editing events to progress to full development in an array of different plant species.

TALE995 can be used to enhance transformation in corn, rice and other plant species. It can be stably maintained on the incoming T-DNA and optionally removed in later stages or in the next generation using genome editing tools, like CRI PSR/Cas9 or TALEN. Alternatively, it may be possible to deliver it transiently; either as episomal-encoded DNA (including viral vectors) or as mRNA/protein. Agrobacterium may be able to deliver the TALE995 transformation booster protein as a fusion with the type IV secretion signal of VirF. The type III secretion system is expected to be able to deliver functional protein as well as TALEs naturally are translocated through this system.

In genome editing the presence of TALE995 may give cells that have received a rare, successful genome edit, a better chance to progress to callus formation, organ formation and eventually seed production. TALE could be delivered transiently or simply be encoded next to genome editing tool coding sequences, like CRISPR/Cas9. After genome editing, the transgene can be segregated along with the genome editing tool genes.

### REFERENCES

H. Scholze and J. Boch (2011). TAL effectors are remote controls for gene activation. Current Opinion in Microbiology 14:47-53
J. Boch, H. Scholze, S. Schornack, A. Landgraf, S. Hahn, S. Kay, T. Lahaye, A. Nickstadt and U. Bonas (2009). Breaking the Code of DNA Binding Specificity of TAL-Type III Effectors. Science 326: 1509-1512
F. Zhang, L. Cong, S. Lodato, S. Kosuri, G. Church and P. Arlotta (2011). Nat Biotechnol. 29(2): 149-153
Christian, M., T. Cermak, E. L. Doyle, C. Schmidt, F. Zhang, A. Hummel, A. J. Bogdanove and D. F. Voytas (2010). “Targeting DNA double-strand breaks with TAL effector nucleases.” Genetics 186(2): 757-761.
Miller, J. C., S. Tan, G. Qiao, K. A. Barlow, J. Wang, D. F. Xia, X. Meng, D. E. Paschon, E. Leung, S. J. Hinkley, G. P. Dulay, K. L. Hua, I. Ankoudinova, G. J. Cost, F. D. Urnov, H. S.

Zhang, M. C. Holmes, L. Zhang, P. D. Gregory and E. J. Rebar (2011). “A TALE nuclease architecture for efficient genome editing.” Nat Biotechnol 29(2): 143-148.
Mussolino, C., R. Morbitzer, F. Lutge, N. Dannemann, T. Lahaye and T. Cathomen (2011). “A novel TALE nuclease scaffold enables high genome editing activity in combination with low toxicity.” Nucleic Acids Res 39(21): 9283-9293.
Sun, N., J. Liang, Z. Abil and H. Zhao (2012). “Optimized TAL effector nucleases (TALENs) for use in treatment of sickle cell disease.” Mol Biosyst 8(4): 1255-1263.
Christian et al., Miller et al., 2011, Mussolino et al., 2011, Sun et al., 2012
Moore et al., ACS Synth Biol. 2014 Oct. 17; 3(10):708-16
Scott et al., FEBS J. 2014 October; 281(20):4583-97.
Zhang et al., Nat Biotechnol. 2011 February; 29(2): 149-153.
Wan et al., Biomed Res Int. 2016; 2016: 8036450.
Cuculis et al., Nat Commun. 2015; 6: 7277
Plant Cell Rep (2007) 26:1567-1573
Mohanta T K, Bashir T, Hashem A, Abd_Allah E F, Bae H. Genome Editing Tools in Plants.
Genes. 2017; 8(12):399.
Winicov et al., Planta (2004) 219: 925-935
Ji et al. Nature Communications volume 7, Article number: 13435 (2016)
Schornack et al. Plant J. 2004 January; 37(1):46-60.
Schornack et al. Molecular Plant-Microbe Interactions 2005 18:11, 1215-1225
Triplett et al. Plant J. 2016 September; 87(5):472-83.
Read et al., Front Plant Sci. 2016 Oct. 13; 7:1516.

1. A method for generating a transgenic plant comprising at least one polynucleotide of interest, the method comprising
(a) providing
(i) a truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
and/or
(ii) a polynucleotide encoding the truncated transcription activator-like effector (TALE) polypeptide of (i), or the functional variant thereof,
(b) providing at least one polynucleotide of interest,
(c) introducing the polypeptide or polynucleotide provided in step (a), and the at least one polynucleotide of interest provided in step (b) into a plant cell, and
(d) regenerating a transgenic plant comprising the at least one polynucleotide of interest from said plant cell.

2. The method of claim 1, wherein the transgenic plant regenerated in step (d) further comprises the polynucleotide encoding the truncated transcription activator-like effector (TALE) polypeptide, or the functional variant thereof.

3. A method for improving plant transformation, the method comprising
(a) providing
(i) a truncated transcription activator-like effector (TALE) polypeptide, or a functional variant of the truncated TALE polypeptide, wherein said truncated TALE polypeptide comprises the N-terminal region of a TALE polypeptide, and optionally a complete or incomplete Central Repeat Region (CRR), and wherein said truncated TALE polypeptide does not comprise a transcriptional activation domain,
and/or
(ii) a polynucleotide encoding the truncated transcription activator-like effector (TALE) polypeptide of (i), or the functional variant thereof,
(b) providing at least one polynucleotide of interest, and
(c) introducing the polypeptide or polynucleotide provided in step (a) and the at least one polynucleotide of interest provided in step (b) into a plant, thereby improving plant transformation.

4. The method of claim 1, wherein said truncated TALE polypeptide does not comprise nuclear localization signals (NLSs).

5. The method of claim 1, wherein said N-terminal region of the TALE polypeptide comprises the T3S and translocation signal and repeats −3, −2, −1 and 0 of a TALE polypeptide.

6. The method of claim 1, wherein the N-terminal region comprises amino acids 1 to 288 of SEQ ID NO: 10.

7. The method of claim 1, wherein said truncated TALE polypeptide comprises an incomplete Central Repeat Region (CRR).

8. The method of claim 7, wherein the incomplete Central Repeat Region (CRR) comprises between 0.5 and 20 repeats, such as between 0.5 and 10 repeats, between 0.5 repeats and 8 repeats, between 0.5 and 6 repeats, between 0.5 and 5 repeats, between 0.5 and 4 repeats, between 0.5 and 3 repeats, between 0.5 and 2 repeats, or between 0.5 and 1 repeats.

9. The method of claim 7, wherein the incomplete Central Repeat Region comprises or consists of the amino acid sequence Itpeqvvaiasnsggkqal (SEQ ID NO:23)

10. The method of claim 9, wherein the truncated TALE polypeptide is truncated within repeats 1 to 20, repeats 1 to 15, repeats 1 to 10, repeats 1 to 8, repeats 1 to 6, repeats 1 to 5, repeats 1 to 4, repeats 1 to 3, repeats 1 to 2, or within repeat 1 of the CRR of the TALE polypeptide.

11. The method of claim 1, wherein the truncated transcription activator-like effector (TALE) polypeptide is derived from a TALE polypeptide from the genus Xanthomonas.

12. The method of claim 11, wherein the truncated transcription activator-like effector (TALE) polypeptide is derived from a TALE polypeptide selected from the group consisting of AvrXa7, AvrBs3, Hax3, PthXo6, AvrXa27, AvrRxo1, PthXo1, Hax2, and Hax4.

13. The method of claim 1, wherein the truncated TALE polypeptide, or functional variant thereof has an amino acid sequence comprising or consisting of
(i) a sequence as shown in any one of SEQ ID Nos: 1, 2, 3, 7, 8, and 9
(ii) a sequence comprising amino acids 1 to 307 of SEQ ID NO: 10
(iii) a sequence comprising amino acids 1 to 443 of SEQ ID NO: 10
(iv) a sequence comprising amino acids 1 to 544 of SEQ ID NO: 10, or
(v) a sequence which is at least 70% identical to the sequence under (i), (ii), (iii) or (iv).

14. The method of claim 1, wherein the polynucleotide encoding said truncated TALE polypeptide, or functional variant thereof, comprises or consists of
(i) a sequence as shown in any one of SEQ ID Nos: 4 to 6, or
(ii) a sequence which is at least 70% identical to the sequence under i).

15. The method of claim 1, wherein the functional variant of the truncated TALE polypeptide is capable of accelerating meristematic plant growth, when introduced into a plant.

16. The method of claim 1, wherein the plant transformation is Agrobacterium-mediated transformation, and wherein in step c) at least the at least one polynucleotide of interest as provided in step b) is stably introduced into the plant by Agrobacterium-mediated transformation.

17. The method of claim 16, wherein i) the truncated TALE polypeptide, or functional variant thereof, or ii) the polynucleotide encoding said truncated polypeptide, or functional variant thereof, is transiently introduced into the plant.

18. The method of claim 17, wherein both the polynucleotide encoding said truncated TALE polypeptide, or functional variant thereof, as provided in step (a)(ii), and the at least one polynucleotide of interest as provided in step b) are stably introduced into the plant by Agrobacterium-mediated transformation.

19. The method of claim 18, wherein the polynucleotide encoding said truncated TALE polypeptide, or functional variant thereof, as provided in step (a)(ii), and the at least one polynucleotide of interest as provided in step b) are present in the same T-DNA or in different T-DNAs.

20. The method of claim 1, wherein the polynucleotide provided in step (a)(ii) and the polynucleotide provided in step (b) are operably linked to a promoter.

21. The method of claim 1, wherein the plant is a monocotyledonous plant.

22. The method of claim 1, wherein the plant is a dicotyledonous plant.

23. The method of claim 3, wherein the improvement of plant transformation is selected from
(i) enhanced transformation efficacy,
(ii) a faster growth of transformed calli,
(iii) a faster generation of TO plants, and
(iv) an increased biomass of generated TO plants.

24. The method of claim 1, wherein the polynucleotide of interest encodes a gene-editing polypeptide.

25. A truncated transcription activator-like effector (TALE) polypeptide, or a functional variant of said truncated TALE polypeptide, wherein said truncated TALE polypeptide comprises the N-terminal region of a TALE polypeptide, and optionally a complete or incomplete TALE Central Repeat Region (CRR), and wherein said truncated TALE polypeptide does not comprise a transcriptional activation domain.

26. A polynucleotide encoding for the truncated TALE polypeptide, or functional variant thereof, of claim 25.

27. An expression vector comprising the polynucleotide of claim 26, operably linked to a heterologous promoter.

28. A host cell comprising the truncated TALE polypeptide of claim 25.

29. The host cell of claim 28, wherein said host cell is an Agrobacterium cell or a plant cell.

30. The host cell of claim 28, further comprising at least one polynucleotide of interest.

31. (canceled)
